SEQUENCE LISTING (3 sequences)

SEQ ID NO: 1 - DNA, 48 bp, Homo sapiens
  source 1..48: mol_type = genomic DNA
gatgtggatcagcaagcaggagtatgacgagtccggcccctccatcgt

SEQ ID NO: 2 - DNA, 52 bp, Homo sapiens
  source 1..52: mol_type = genomic DNA
ccaccgcaaatgcttctaggcggactatgacttagttgcgttacaccctttc

SEQ ID NO: 3 - DNA, 33 bp, synthetic construct
  source 1..33: mol_type = other DNA
ggcggctgttcaagaagattagctagtcggact

(19) United States
(12) Patent Application Publication
(10) Pub. No.: US 2025/0109388 A1
(43) Pub. Date: Apr. 3, 2025

(54) METHOD FOR ISOLATING AND CONCENTRATING EXTRACELLULAR VESICLE GROUP DERIVED FROM CANCER CELL, ANALYSIS METHOD, DRUG SELECTION METHOD, KIT, AND ANTI-CANCER AGENT

(71) Applicant: THE UNIVERSITY OF TOKYO, Tokyo (JP)

(72) Inventors: Motoyuki OTSUKA, Tokyo (JP); Mitsuhiro FUJISHIRO, Tokyo (JP); Chikako SHIBATA, Tokyo (JP)

(73) Assignee: THE UNIVERSITY OF TOKYO, Tokyo (JP)

(21) Appl. No.: 18/857,897

(22) PCT Filed: Apr. 25, 2023
(86) PCT No.: PCT/JP2023/016313

§ 371 Date: Oct. 18, 2024

Related U.S. Application Data
(60) Provisional application No. 63/334,672, filed on Apr. 26, 2022.

(51) Int. Cl. C12N 5/09; C07K 16/30; C12Q 1/6886

(52) U.S. Cl. CPC C12N 5/0693; C07K 16/303; C12Q 1/6886; C12Q 2600/106

(57) ABSTRACT

A method for isolating and concentrating an extracellular vesicle group derived from a cancer cell involves contacting a substance which has affinity towards a carbohydrate antigen CA19-9 with an extracellular vesicle group which has been isolated from blood derived from a patient, and isolating and concentrating an extracellular vesicle group.

### TECHNICAL FIELD

The present invention relates to a method for isolating and concentrating an extracellular vesicle group derived from a cancer cell, an analysis method, a drug selection method, a kit, and an anti-cancer agent.

### BACKGROUND ART

Research has been being promoted worldwide to isolate extracellular vesicles (EVs) in the blood and analyze the contents thereof for the purpose of the diagnosis of diseases.

Cancer-associated cachexia (CAC) is a wasting state that is frequently observed in patients with advanced cancer, particularly pancreatic cancer. CAC is a multifactorial disease, and one of the features thereof is systemic lipolysis (see, for example, Non-Patent Document 1). The development of intervention methods requires a deeper understanding of the molecular basis of the lipolysis in CAC. It is considered that the analysis of extracellular vesicles (EVs) released from cancer cells can contribute to the development of the intervention methods for CAC.

### PRIOR ART REFERENCES

Non-Patent Document 1: Fearon, K. C. H., Glass, D. J. & Guttridge, D. C. Cancer Cachexia: Mediators, Signaling, and Metabolic Pathways. Cell Metabolism 16, 153-166, doi:10.1016/j.cmet.2012.06.011 (2012).

### SUMMARY OF THE INVENTION

### Problems to be Solved by the Invention

However, it is assumed that the lack of sensitivity and accuracy may occur in a case of collectively analyzing extracellular vesicles in the blood, which is a miscellaneous population. On the other hand, it is difficult to specifically isolate and concentrate only extracellular vesicles that are released from cells, organs, or the like of patients suffering from specific lesions, and thus there is a demand for a new method for isolating and concentrating a specific subset of extracellular vesicles derived from organs, cells, or the like, from a group of extracellular vesicles in the blood.

The present invention has been made in consideration of the above circumstances and provides a method for isolating and concentrating an extracellular vesicle group derived from a cancer cell, which makes it possible to diagnose cancer in a highly accurate and noninvasive manner, an analysis method, a drug selection method, a kit, and an anti-cancer agent.

### Means for Solving the Problems

The present invention includes the following aspects.

[1] A method for isolating and concentrating an extracellular vesicle group derived from a cancer cell, the method including:
bringing a substance having an affinity for a carbohydrate antigen CA19-9 into contact with an extracellular vesicle group which has been isolated from blood derived from a patient, and isolating and concentrating an extracellular vesicle group derived from a cancer cell, from the extracellular vesicle group.

[2] The method for isolating and concentrating an extracellular vesicle group derived from a cancer cell according to [1], in which the substance having an affinity for a carbohydrate antigen CA19-9 is an anti-CA19-9 antibody.

[3] The method for isolating and concentrating an extracellular vesicle group derived from a cancer cell according to [1], in which the substance having an affinity for a carbohydrate antigen CA19-9 further includes a carrier.

[4] The method for isolating and concentrating an extracellular vesicle group derived from a cancer cell according to [3], in which the carrier is a bead.

[5] An analysis method including:
analyzing cancer-associated genes in an extracellular vesicle group derived from a cancer cell by the method according to any one of [1] to [4], for a genotype of the patient.

[6] A drug selection method including:
selecting a drug to be administered, based on the genotype of the patient, which is analyzed using the analysis method according to [5].

[7] A kit for isolating and concentrating an extracellular vesicle group derived from a cancer cell, from an extracellular vesicle group isolated from blood derived from a patient, the kit containing:
a substance having an affinity for a carbohydrate antigen CA19-9.

[8] The kit according to [7], in which the substance having an affinity for a carbohydrate antigen CA19-9 is an anti-CA19-9 antibody.

[9] The kit according to [7], in which the substance having an affinity for a carbohydrate antigen CA19-9 includes a carrier.

[10] The kit according to [9], in which the carrier is a bead.

[11] An anti-cancer agent containing:
as an active ingredient, an anti-CA19-9 antibody that neutralizes a function of a carbohydrate antigen CA19-9.

### Effects of the Invention

According to the present invention, it is possible to diagnose cancer in a highly accurate and noninvasive manner.

### BRIEF DESCRIPTION OF DRAWINGS

FIG. 1 This figure shows results of Western blotting of extracellular vesicle markers (CD63 and TSG101) in 0.8 μg of EV (left panel) from a culture medium for cell culture and 1.5 μg of EV (right panel) from the mixed serum as healthy controls (n=5) and the mixed serum of pancreatic cancer patients (n=5).

FIG. 2 This figure is a view that shows a method differentiating human adipocyte-derived mesenchymal stem cells (hAD-MSCs) into adipocytes.

FIG. 3 This figure shows an image of hAD-MSC and differentiated adipocytes, which are obtained by staining with Oil Red O. Scale bar: 20 μm. The level of Oil red O in cells, which is determined by colorimetric analysis, is shown. Data are in terms of mean±SD (n=3). **P=0.00020.

FIG. 4 This figure shows graphs that show expression of adipocyte markers after differentiation. mRNA levels of C/EBPα, FABP4, HSL, and GLUT4 in hAD-MSC and differentiated adipocytes, which are obtained by RT-qPCR, are shown. Data are in terms of mean±SD (n=3). **P=0.0028 (C/EBPα). **P=0.00023 (FABP4), **P=0.0021 (HSL), and **P=0.00065 (GLUT4). Statistical analyses were carried out using Welch's t-test.

FIG. 5 This figure is a graph that shows glycerol release from human adipocytes treated for 24 hours with EVs (1.5 μg) derived from the indicated cells. PBS was used as a negative control. Data are in terms of mean±SD (n=4). **P=0.0011 (Panc-1), **P=0.00019 (Miapapaca-2), **P=0.0013 (BxPC-3), and P=0.99 (Capan-2).

FIG. 6 This figure is a view that shows results of Western blotting of factors associated with intracellular lipolytic signaling in lysates of human adipocytes treated for 1 hour with EVs (1.5 μg) derived from the indicated cells. Representative images from three independent experiments are shown. A band intensity of phosphorylated HSL (pHSL; HSL phosphorylated at Ser660) with respect to the total HSL protein level, as well as a band intensity of the total HSL and a band intensity of ATGL with respect to the D-actin protein level are shown at the bottom of the panel.

FIG. 7 This figure is a graph that shows a cAMP level of adipocytes treated for 1 hour with EV (1.5 μg). Data are in terms of mean±SD (n=3). **P=0.0023 (Panc-1), **P=0.0019 (Miapapaca-2), **P=0.00023 (BxPC-3), and P=0.11 (Capan-2).

FIG. 8 is a graph that shows glycerin release from human adipocytes treated with EVs (1.5 g) derived from Panc-1 cells, in the presence and absence of 10 μM H89 (a PKA inhibitor), 0.5 μM orlistat (OLST, an inhibitor for HSL and ATGL), and 0.1 M Cay10499 (an HSL inhibitor).

FIG. 9 (A) is a graph that shows glycerin release from adipocytes treated with 10 μM isoproterenol (ISO), in the presence or absence of a 24-hour treatment with 30 μM SQ22536 (SQ, an adenylyl cyclase inhibitor). Data are in terms of mean±SD (n=3). **P=0.0025. (B) is a graph that shows glycerin release from adipocytes treated with EVs (1.5 g) derived from Panc-1 cells, in the presence or absence of a 24-hour treatment with 30 μM SQ22536. Data are in terms of mean±SD (n=3). P=0.65. ns indicates that no significant difference is observed. P=0.65.

FIG. 10 This figure shows immunofluorescence images of adipocytes treated for 5 hours with PBS and EV (1.5 μg) from Panc-1 and Capan-2 culture media. The membrane of the EV was labeled with Exosparkler. The nuclei were stained with DAPI. Representative images from three independent experiments are shown. Scale bar: 30 μm. Statistical analyses were carried out using Welch's t-test.

FIG. 11 This figure shows results of Western blotting of the β-actin protein in EVs derived from pancreatic cancer cells. Representative results of Western blotting of the β-actin level in EVs (0.8 μg) derived from pancreatic cancer cells in three independent experiments are shown. The band intensity is shown with the level of EVs derived from BxPC-3 cells adjusted to 1.

FIG. 12 This figure is a view that shows a method for establishing Panc-1 cells having a HiBiT sequence downstream of the ACTB gene. The HiBiT peptide sequence was inserted immediately before the stop codon of the ACTB gene by Cas9-mediated gene editing to generate β-actin bound to the endogenous HiBiT peptide.

FIG. 13 (A) shows results of Western blotting of β-actin-HiBiT in wild-type cells and ACTB-HiBiT cells. A representative graph obtained from three independent experiments is shown. The relative band intensity of the HiBiT level corrected by β-actin is shown. Data are in terms of mean±SD (n=3). **P<104. (B) is a graph that shows luciferase levels in wild-type Panc-1 cells (Wild-type) and Panc-1-ACTB-HiBiT cells (left panel) and luciferase levels in the wild-type cells and EV particles (right panel) of 10⁹ particles from the ACTB-HiBiT cells. Data are in terms of mean±SD (n=4). **P<10⁻⁴ (cell lysate), and **P<10⁻⁵ (EV).

FIG. 14 This figure shows results of the internalization of EVs added to human adipocytes, where the internalization thereof is determined by the luciferase activity of β-actin-HiBiT in EVs derived from Panc-1 cells. The luciferase level is a luciferase level 5 of adipocytes treated for 1 hour with EV (1.5 g) in the presence or absence of a treatment with an endocytosis inhibitor (20 IM chlorpromazine (CPZ), 10 μM nocodazole, 20 μM cytochalasin D (CytD), or 2.5 μg/mL nystatin, or incubation at 4° C.), which is a value indicated in terms of comparison with a negative control (PBS). Data are in terms of mean±SD (n=3). P=0.74 (CPZ), *P=0.029 (nocodazole), *P=0.032 (CytD), **P=0.0078 (nystatin), and **P=0.0074 (4° C.); comparison with cells treated with EV alone (control).

FIG. 15 This figure is a graph of glycerin release from adipocytes treated for 24 hours with EVs (1.5 μg) derived from Panc-1 cells, in the presence or absence of an inhibitor of endocytosis. The dosage of the inhibitor is as described in FIG. 14. Data are in terms of mean±SD (n=3). P=0.40 (CPZ), *P=0.027 (Nocodazole), *P=0.012 (CytD). **P=0.0065 (Nystatin), and **P=0.0074 (4° C.); comparison with cells treated with EV alone (control). Statistical analyses were carried out using Welch's t-test.

FIG. 16 (A) shows a graph of glycerin release from human adipocytes treated for 24 hours with 2.5 μL of EVs derived from the pooled sera from 5 persons as healthy controls (normal) or 5 patients as pancreatic cancer patients (cancer). PBS was used as a negative control. Data are in terms of mean±SD (n=3). *P=0.044 (normal), **P=0.00077 (cancer), and *P=0.032 (normal versus cancer). (B) shows results of Western blotting of factors associated with intracellular lipolytic signaling in a lysate of human adipocytes treated for 1 hour with 2.5 μl of EVs from the pooled sera from 5 persons as healthy controls (normal) or 5 patients as pancreatic cancer patients (cancer). A representative graph obtained from three independent experiments is shown. A band intensity of phosphorylated HSL (pHSL) with respect to the total HSL protein level as well as a band intensity of the total HSL and a band intensity of ATGL with respect to the β-actin protein level are shown at the bottom of the panel. Data are in terms of mean±SD (n=3). For pHSL, P=0.87 (normal), and **P<10⁻⁵ (cancer); ns indicates that no significant difference is observed.

FIG. 17 This figure is a graph that shows a cAMP level of adipocytes treated for 1 hour with EVs from 2.5 μL of pooled sera from 5 persons as healthy controls (normal) or 5 patients as pancreatic cancer patients (cancer). Data are in terms of mean±SD (n=3). *P=0.015 (normal), **P=0.0033 (cancer), and *P=0.023 (normal versus cancer).

FIG. 18 This figure is a graph that shows a cAMP level of EVs from 2.5 μL of pooled sera from 5 persons as healthy controls (normal) or 5 patients as pancreatic cancer patients (cancer).

FIGS. 19 (A) and (B) show graphs glycerin release from adipocytes treated for 24 hours with EV of 10⁹ particles ((A)) or 1 μg of EV ((B)), from the pooled sera from 5 persons as healthy controls (normal) or 5 patients as pancreatic cancer patients (cancer). PBS was used as a control. Data are in terms of mean±SD (n=3). **P<0.01. The P value for the difference between normal and cancer is as follows: **P<10⁻⁴ (A), and *P=0.0022 (B). Statistical analyses were carried out using Welch's t-test.

FIG. 20 This figure is a table that shows the clinical features of the patients included in the test in the present application.

FIG. 21 (A) is a graph of glycerin release from human adipocytes treated for 24 hours with 1 μg of EVs (normal EVs; n=10) derived from serum from healthy controls and 1 μg of EVs (cancer EVs: n=10) derived from serum from pancreatic cancer patients. Data are in terms of mean±standard deviation. *P=0.017. (B) is a graph that shows a cAMP level of 1 μg of EVs derived from each of the sera of the healthy control (normal EVs; n=10) and pancreatic cancer patients (cancer EVs: n=10). The data are in terms of mean±SD. ns indicates that no significant difference is observed. P=0.29

FIGS. 22 (A) and (B) are graphs that shows a correlation between the glycerin release from the EV-treated adipocytes and the cAMP level of EV ((A)) or a correlation between the glycerol release and the mode size of serum EV ((B)). The circles in the graph show EVs (filled circles: n=10) derived from the healthy controls and EVs (open circles: n=10) derived from the pancreatic cancer patients, respectively. The broken line is a regression line. Correlations were estimated according to Pearson's correlation coefficients.

FIG. 23 This figure is a graph of the mode size of EVs derived from healthy controls (normal EVs; n=10) and pancreatic cancer patients (cancer EVs; n=19). Data are in terms of mean t standard deviation. *P=0.014. Statistical analyses were carried out using Welch's t-test.

FIG. 24 This figure is a view that shows a procedure for changing the size of EV. EV was isolated from a high glucose culture medium or low glucose culture medium of Panc-1 cells. EV-high glucose (EV-H): EVs derived from high glucose culture, EV-low glucose (EV-L): EVs derived from low glucose culture.

FIG. 25 This figure shows graphs showing the size distributions of EV-H and EV-L. The size of the EV-L was smaller. Representative results from at least three independent experiments are shown.

FIG. 26 This figure shows results of Western blotting of the β-actin protein levels in 10⁹ particles of EVs (EV-H and EV-L from Panc-1 cells). Representative images from three independent experiments are shown. A band intensity of β-actin with respect to the EV marker CD9 is shown. Data are in terms of mean±SD (n=3). ns indicates that no significant difference is observed. P=0.24.

FIG. 27 (A) is a graph that shows relative luciferase levels reflecting the level of β-actin-HiBiT in 10 particles of EV. Wild type: EVs derived from Panc-1 cells (not expressing HiBiT) as a negative control, EV-H and EV-L: EVs derived from Panc-1-ACTB-HiBiT cells cultured with high glucose and low glucose, respectively. Data are in terms of mean±SD (n=4). **P<104 (EV-H), and **P<10⁻⁵ (EV-L). ns indicates that no significant difference is observed. P=0.61 (EV-H versus EV-L). (B) is a graph that shows cAMP levels of 10⁹ particles of EV-H and EV-L. Data are in terms of mean f SD (n=4). ns indicates that no significant difference is observed. P=0.72. (C) is a graph that shows the level of the internalization of EV into human adipocytes. The relative luciferase level of adipocytes treated for 1 hour with 10⁹ particles of EVs (EV-H and EV-L) derived from Panc-1-ACTB-HiBiT cells was compared with the negative control (PBS). Data are in terms of mean±SD (n=3). **P=0.0011.

FIG. 28 This figure shows immunofluorescence images of human adipocytes treated for 1 hour with PBS and EVs (EV-H and EV-L) derived from the culture medium for the Panc-1 cell culture. The EV membrane was labeled with Exosparkler. The nuclei were stained with DAPI. Scale bar: 50 μm.

FIG. 29 This figure is a graph that shows glycerol release from human adipocytes treated for 24 hours with 10⁹ particles of EVs (EV-H and EV-L) derived from Panc-1 cells. PBS was used as a negative control. Data are in terms of mean±SD (n=3). **P=0.00056 (EV-H). **P=0.00026 (EV-L), and *P=0.015 (EV-H versus EV-L). Statistical analyses were carried out using Welch's t-test.

FIG. 30 This figure is a view that shows a scheme for treating EV with NP40. EV-NP40: EV was isolated from the culture solution, and then the EV was treated with NP40, followed by purification with a qEV column.

FIG. 31 This figure shows graphs that show the size distributions of EV and EV-NP40 from the culture medium for the Panc-1 cell culture. Representative results from at least three independent experiments are shown in terms of mode and mean.

FIG. 32 (A) is a graph that shows luciferase levels as an indicator of the β-actin-HiBiT protein level in EV. Wild type; Panc-1 cells (not expressing HiBiT) as a negative control, EV and EV-NP40; EVs derived from Panc-1-ACTB-HiBiT cells. 10⁹ particles per sample were used. Data are in terms of mean±SD (n=3). **P=0.0017 (EV), **P<104 (EV-NP40), and **P=0.0026 (EV versus EV-NP40). (B) is a graph that shows the internalization of EV into human adipocytes. The luciferase level of the lysate of adipocytes treated for 1 hour with EVs (10⁹ particles) derived from Panc-1-ACTB-HiBiT cells or EV-NP40 was compared with the negative control (PBS), and the results are shown. Data are in terms of mean±SD (n=3). *P=0.045. Statistical analyses were carried out using Welch's t-test.

FIG. 33 (A) is a graph that shows a CA19-9 level measured using ELISA. The CA19-9 level either in 1 mL of the pooled sera (left panel) or in EVs (right panel) derived from 1 ml of the pooled sera, from 5 persons as healthy controls (normal) or 5 patients as pancreatic cancer patients (cancer), is shown. Data are in terms of mean±SD (n=2 for serum. n=4 for EV). **P=0.00050 (serum), and **P<10⁻⁶ (EV). (B) shows results of Western blotting of CA19-9 in 0.2 μg of EV from the pooled sera from 5 persons as healthy controls (normal) or 5 patients as pancreatic cancer patients (cancer).

FIG. 34 This figure is a graph that shows the CA19-9 level of EVs (1 mg) derived from normal human pancreatic ductal epithelial cells (HPDE and HPNE) and pancreatic cancer cell lines (Panc-1, Miapaca-2, BxPC-3, and Capan-2), which is determined by ELISA. EVs derived from normal human pancreatic ductal epithelial cells, Panc-1, and Miapaca-2 cells rarely express CA19-9. Data are in terms of mean f SD (n=4). ns indicates that no significant difference is observed. **P<10⁻⁵.

FIG. 35 This figure shows representative results of Western blotting of the CA19-9 level in cell lysates and EVs (0.8 μg) from the indicated cell lines. Representative images from three independent experiments are shown. The band intensity of CA19-9 with respect to β-actin (cell lysate) or CD9 (EV) is shown in the bottom figure. Data are in terms of mean±SD (n=3).

FIG. 36 This figure shows immunofluorescence staining images in Panc-1 (CA19-9 negative) and BxPC-3 (CA19-9 positive) cells. The nuclei were stained with DAPI. Representative images from three independent experiments are shown. Scale bar: 10 μm. Statistical analyses were carried out using Welch's t-test.

FIG. 37 This figure shows results obtained by subjecting the EV (3 μg) immunoprecipitated using an anti-CA19-9 antibody to Western blotting using the anti-CA19-9 antibody and an anti-AFP antibody. An isotype IgG thereof was used as a control. The input was 2% of EV before immunoprecipitation. The EVs derived from Huh7 cells were AFP positive and CA19-9 negative (liver cancer cells). The EVs derived from Capan-2 cells were AFP negative and CA19-9 positive (pancreatic cancer cells). Representative images from at least three independent experiments are shown.

FIG. 38 (A) is a graph that shows the frequency of KRAS mutations (KRAS mut/WT ratio: ratio of frequency of mutant-type KRAS RNA to wild-type KRAS RNA) according to ddPCR in RNA in EVs derived from Huh7 cells and EVs derived from Capan-2 cells. Huh7 has a wild-type KRAS gene, and Capan-2 has a heterozygous mutant KRAS gene. Data are in terms of mean±SD (n=3). (B) is a graph that shows the frequency of KRAS mutations according to ddPCR in RNA of EVs derived from Huh7 cells and Capan-2 cells, which are pooled in a 1:1 ratio. The frequency of KRAS mutations was determined regardless of the presence or absence of preamplification PCR. Data are in terms of mean±SD (n=3). ns indicates that no significant difference is observed. P=0.55.

FIG. 39 This figure shows results of the analysis of the frequency of KRAS mutations according to ddPCR in RNA of the EVs which have been immunoprecipitated (IP) using a CA19-9 antibody from a 1:1 mixture of EVs (a sample before IP) derived from Huh7 cells and Capan-2 cells. Data are in terms of mean±SD (n=3). **P=0.0080.

FIG. 40 This figure shows results of isolating, by IP targeting CA19-9, EVs derived from pancreatic cancer, from the patient serum. The EVs (10 μg) derived from the pooled sera from 5 persons as healthy controls (normal) or 5 patients as pancreatic cancer patients (cancer) were subjected to IP. An aliquot from each sample before IP (2%) was used as an input. An anti-CA19-9 antibody was used for IP, and an isotype IgG thereof was used as a negative control. Representative results of Western blotting of CA19-9 in three independent experiments are shown. NS indicates a non-specific band.

FIG. 41 This figure shows results of ddPCR regarding the frequency of KRAS mutations in RNA of the EVs derived from the pooled sera (before IP) from the pancreatic cancer patients or the cancer-derived EVs from the pooled sera, which have been captured by anti-CA19-9 beads (IP). Data are in terms of mean±SD (n=3). *P=0.026.

FIG. 42 This figure shows results of the size distributions of EVs (left panel. input) derived from Huh7 cells and EVs (right panel. IP and elution) derived from Huh7 cells, which have been immunoprecipitated using an anti-CD63 antibody and then eluted. Representative results from at least three independent experiments are shown in terms of mode and mean.

FIG. 43 This figure shows results of a size distribution (left panel, input) of EVs isolated from the pooled sera from 5 pancreatic cancer patients and a size distribution (right panel. IP and elution) of pancreatic cancer-specific EVs which have been immunoprecipitated with an anti-CA19-9 antibody from bulk EVs and then eluted. Representative results from at least three independent experiments are shown in terms of mode and mean. Statistical analyses were carried out using Welch's t-test.

FIG. 44 This figure shows a schematic view of an in vivo experiment. EV-H and EV-L (5×10¹⁰ particles) from the culture solution of Panc-1-ACTB-HiBiT cells, or PBS (control) were administered to the tail veins of BALB/c mice (n=3 (PBS), n=4 (EV-H), and n=4 (EV-L) twice weekly for 4 weeks.

FIG. 45 This figure is a graph that shows body weight changes in mice from baseline in a period of 4 weeks after the injection. The data are in terms of mean±SD. P=0.13 (EV-H), *P=0.047 (EV-L), and *P=0.037 (EV-H versus EV-L).

FIG. 46 This figure is a graph that shows changes in the average body weights.

FIG. 47 This figure is a graph that shows the body weight of the gWAT at 4 weeks after the injection. The data are in terms of mean±SD. P=0.39 (EV-H), *P=0.028 (EV-L), and P=0.22 (EV-H versus EV-L).

FIG. 48 This figure shows representative photographic images of the abdominal organs at 4 weeks after injection. The broken line indicates the gWAT.

FIG. 49 This figure is a graph that shows the distribution of EVs after the injection, which is estimated from the luciferase activity of the tissue extract. The relative luciferase level (n=3) of the gWAT is shown. The data are in terms of mean f SD. P=0.072 (EV-H), **P=0.00025 (EV-L), and **P=0.00027 (EV-H versus EV-L).

FIG. 50 This figure shows representative graphs that show the amounts of internalized EVs, which are estimated by the luciferase activity in the serum, lung, and the muscle of the mouse. Data are in terms of mean±SD (n=3). ns indicates that no significant difference is observed. *P<0.05; and **P<0.01.

FIG. 51 This figure shows representative images of the gWAT according to H&E staining. Scale bar: 50 μm. The size distribution of fat droplets is shown in the bottom figure. The sizes of 300 fat droplets were analyzed in three mice per group.

FIG. 52 This figure shows results of Western blotting of factors associated with intracellular lipolytic signaling in the lysates of the gWAT at 4 weeks after the injection. Representative images from two mice per group are shown. A band intensity of pHSL with respect to the total HSL protein as well as a band intensity of the total HSL and a band intensity of ATGL with respect to the R-actin protein are shown in the bottom figure. Data are in terms of mean±SD (n=3). Statistical analyses were carried out using Welch's t-test.

FIG. 53 This figure shows a conceptual view of the detection of surface proteins of EV using an oligo DNA.

FIG. 54 This figure shows results of quantitative PCR for the TF.

### DETAILED DESCRIPTION FOR CARRYING OUT THE EMBODIMENTS

<<Method for Isolating and Concentrating Extracellular Vesicle Group Derived from Cancer Cell>>

The present embodiment provides a method for isolating and concentrating an extracellular vesicle group derived from a cancer cell, comprising:
bringing a substance having an affinity for a carbohydrate antigen CA19-9 into contact with an extracellular vesicle group which has been isolated from blood derived from a patient, and isolating and concentrating an extracellular vesicle group derived from a cancer cell, from the extracellular vesicle group.

As will be described later in Examples, the inventors of the present invention have found that carbohydrate antigen CA19-9 is enriched in EVs isolated from pancreatic cancer patients.

The carbohydrate antigen CA19-9 is a sialyl Lea antigen that is recognized by a mouse monoclonal antibody NS19-9 and is known as one of the tumor markers.

The cancers of interest are not particularly limited, and examples thereof include, but not limited to, breast cancer (for example, invasive ductal carcinoma, non-invasive ductal carcinoma, or inflammatory breast cancer), prostate cancer (for example, hormone-dependent prostate cancer or hormone-independent prostate cancer), pancreatic cancer (for example, pancreatic ductal carcinoma), gastric cancer (for example, papillary adenocarcinoma, mucous adenocarcinoma, or adenosquamous carcinoma), lung cancer (for example, non-small cell lung cancer, small cell lung cancer, or malignant mesothelioma), colorectal cancer (for example, gastrointestinal stromal tumor), rectal cancer (for example, gastrointestinal stromal tumor), colon cancer (for example, familial colorectal cancer, hereditary non-polyposis colon cancer, or gastrointestinal stromal tumor), small intestine cancer (for example, non-Hodgkin lymphoma or gastrointestinal stromal tumor), esophageal cancer, duodenal cancer, tongue cancer, throat cancer (for example, nasopharyngeal cancer, oropharyngeal cancer, or hypopharyngeal cancer), head and neck cancer, salivary gland cancer, a brain tumor (for example, pineal astrocytic tumor, pilocytic astrocytoma, diffuse astrocytoma, or anaplastic astrocytoma), neurilemmoma, liver cancer (for example, primary liver cancer or extrahepatic bile duct cancer), kidney cancer (for example, renal cell carcinoma or transitional cell carcinoma of the renal pelvis and ureter), gallbladder cancer, pancreatic cancer, endometrial cancer, cervical cancer, ovarian cancer (for example, epithelial ovarian cancer, extragonadal germ cell tumor, ovarian germ cell tumor, or ovarian low malignant potential tumor), bladder cancer, urethral cancer, skin cancer (for example, intraocular (eye) melanoma or Merkel cell cancer), hemangioma, malignant lymphoma (for example, reticulosarcoma, lymphosarcoma, or Hodgkin's disease), melanoma (malignant melanoma), thyroid cancer (for example, medullary thyroid cancer), parathyroid cancer, nasal cavity cancer, paranasal sinus cancer, a bone tumor (for example, osteosarcoma. Ewing's tumor, uterine sarcoma, or soft tissue sarcoma), metastatic medulloblastoma, angiofibroma, dermatofibrosarcoma protuberans, retinoblastoma, penile cancer, testicular tumor, a pediatric solid tumor (for example, Wilms tumor or a pediatric renal tumor), Kaposi's sarcoma, AIDS-related Kaposi's sarcoma, tumor of maxillary sinus, fibrous histiocytoma, leiomyosarcoma, rhabdomyosarcoma, myeloproliferative disease, and leukemia (for example, acute myeloid leukemia or acute lymphoblastic leukemia).

In the isolation and concentration method according to the present embodiment, the substance having an affinity for a carbohydrate antigen CA19-9 may be any substance as long as it has an affinity for a carbohydrate antigen CA19-9. Examples thereof include mucin and an anti-CA19-9 antibody, among which an anti-CA19-9 antibody is preferable. Examples of the antibody include a monoclonal antibody, a polyclonal antibody, a multispecific antibody (for example, a bispecific antibody), and an antibody fragment.

It is preferable that the substance having an affinity for a carbohydrate antigen CA19-9 further includes a carrier. Examples of the carrier include beads of silicon, titanium dioxide, aluminum oxide, glass, polystyrene, cellulose, polyamide, and the like.

An example of the isolation and concentration method according to the present embodiment specifically includes the following steps. First, the serum that is isolated from the blood derived from the patient is centrifuged at 2,000×g for 4° C. for 10 minutes, and then the supernatant is filtered using a 0.45 μm filter. Next, the supernatant is applied onto a size exclusion column to obtain an extracellular vesicle group.

The extracellular vesicle group obtained is brought into contact with protein A/G magnetic beads to which an anti-CA19-9 antibody is bound, and then an extracellular vesicle group derived from cancer cells is isolated and concentrated from the extracellular vesicle group by a general immunoprecipitation method.

Further, the precipitate substance obtained using the anti-CA19-9 antibody may be labeled with a label for a molecule that is highly expressed in the CA19-9 positive EV. The label is not particularly limited, and examples of the method for labeling include a method that uses, for labeling, a labeled antibody against the corresponding molecule. Examples of the substance that is used for labeling an antibody include labeling enzymes, examples of which include horseradish peroxidase and alkaline phosphatase

In addition, as will be described later in Examples, an antibody obtained by binding an oligonucleotide to an antibody against the corresponding molecule may be used to carry out labeling. By carrying out quantitative PCR for this oligonucleotide, the expression level of the corresponding molecule on the EV can be quantified.

### <Analysis Method>>

The present embodiment provides a method for analyzing cancer-associated genes in an extracellular vesicle group derived from a cancer cell to analyze a genotype of the patient, where the extracellular vesicle group is isolated and concentrated by using the above-described method for isolating and concentrating an extracellular vesicle group derived from a cancer cell.

As will be described later in Examples, by using the isolation and concentration method according to the present embodiment, it is possible to isolate and concentrate EVs derived from cells having a KRAS mutation. Cancer-associated genes of the isolated and concentrated extracellular vesicle group derived from cancer cells are analyzed using, for example, a next-generation sequencer.

Cancer genes include a group of genes encoding a growth factor, such as sis; a group of genes encoding a receptor type tyrosine kinase, such as erbB, fins, and ret; a group of genes encoding a non-receptor type tyrosine kinases, such as fes; a group of genes encoding a GTP/GDP-binding protein, such as ras; a group of genes encoding a serine/threonine kinase, such as src, mos, or raf; a group of genes encoding a nuclear protein, such as myc, myb, fos, jun, or erbA; a group of genes encoding a signal transduction adapter molecule, such as crk; and a fused gene such as Bcr-Abl.

Further, examples of the cancer genes include genes associated with the Ras-MAP kinase pathway, such as Shc, Grb2, Sos, MEK, Rho, and Rac genes; genes associated with the phospholipase C gamma-protein kinase C pathway, such as PLCY and PKC: genes associated with the PI3K-AKT pathway, such as PI3K, Akt, and Bad: genes associated with the JAK-STAT pathway, such as JAK and STAT; and genes associated with the GAP-based pathway, such as GAP, p¹80, and p62.

In addition, mutations in tumor suppressor genes such as p53, Rb, and BRCA1 may be analyzed.

### <<Drug Selection Method>>

The present embodiment provides a drug selection method for selecting a drug to be administered, based on the genotype of the patient, which is analyzed using the above-described analysis method.

For example, in a case where the genotype of EVs derived from a patient after cancer chemotherapy has been determined to be positive for the KRAS G12C mutation by using the analysis method according to the present embodiment, a KRAS inhibitor can be selected. The drug selection method according to the present embodiment is noninvasive since it uses EVs isolated from the blood.

### <<Kit>>

The present embodiment provides a kit for isolating and concentrating an extracellular vesicle group derived from a cancer cell, from an extracellular vesicle group isolated from blood derived from a patient, where the kit contains a substance having an affinity for a carbohydrate antigen CA19-9. The constitution of the kit is the same as that described in <<Method for isolating and concentrating extracellular vesicle group derived from cancer cell>>.

### <<Anti-Cancer Agent>>

The present embodiment provides an anti-cancer agent, containing as an active ingredient, an anti-CA19-9 antibody that neutralizes a function of a carbohydrate antigen CA19-9.

The anti-cancer agent according to the present embodiment can be also administered, for example, orally in a form of a tablet, a coated tablet, a pill, a powdered drug, a granule, a capsule agent, a liquid agent, a suspension, or an emulsion, or parenterally in a form of an injection agent, a suppository, or a skin external agent.

As the pharmaceutically acceptable carrier, a carrier that is used for a general pharmaceutical preparation can be used without particular limitation. More specific examples thereof include binding agents such as gelatin, corn starch, tragacanth gum, and gum arabic; excipients such as starch and crystalline cellulose; swelling agents such as alginic acid; solvents for an injection agent, such as water, ethanol, and glycerin; and pressure-sensitive adhesives such as a rubber-based pressure-sensitive adhesive and a silicone-based pressure-sensitive adhesive. One kind of pharmaceutically acceptable carrier can be used singly, or two or more kinds thereof can be mixed and used.

The anti-cancer agent according to the present embodiment may further contain additives. Examples of the additive include lubricants such as calcium stearate and magnesium stearate; sweetening agents such as sucrose, lactose, saccharin, and maltitol; flavoring agents such as peppermint and Gaultheria adenothrix oil; stabilizers such as benzyl alcohol and phenol; buffering agents such as a phosphoric acid salt and sodium acetate; dissolution assisting agents such as benzyl benzoate and benzyl alcohol; antioxidants; and preservatives.

The additive can be used alone, or two or more thereof can be mixed and used.

### (Administration Method)

The administration method for the anti-cancer agent according to the present embodiment is not particularly limited and may be appropriately determined depending on the symptom, body weight, age, sex, and the like of a patient. For example, a tablet, a coated tablet, a pill, a powdered drug, a granule, a capsule agent, a liquid agent, a suspension, or an emulsion is administered orally. In addition, an injection agent is intravenously administered singly or as a mixture with a general replacement fluid such as glucose or amino acids, and as necessary, is administered intraarterially, intramuscularly, intracutaneously, subcutaneously, or intraperitoneally.

### (Dosage)

The dosage of the anti-cancer agent according to the present embodiment varies depending on the symptom, body weight, age, sex, and the like of the patient, and thus it cannot be determined unconditionally. However, in a case of oral administration, for example, 1 μg to 10 g per day, for example, 0.01 to 2,000 mg per day in terms of the active ingredient may be administered. In addition, in a case of an injection agent, for example, 0.1 μg to 1 g per day, for example, 0.001 to 200 mg per day in terms of active ingredient may be administered. In addition, in a case of a suppository, for example, 1 μg to 10 g per day, for example, 0.01 to 2,000 mg per day in terms of the active ingredient may be administered.

### EXAMPLES

The present invention will be described with reference to Examples; however, the present invention is not limited to Examples below.

CAC is a tumor-associated syndrome that is characterized by body weight loss, skeletal mass wasting, and adipose tissue atrophy. Although CAC occurs in the majority of cancer patients, it often appears in the early stage of the pancreatic cancer which has a high mortality rate. Although agonists for ghrelin receptor are currently used in hospitals in order to ameliorate anorexia in CAC, a new therapy is needed for this multifactorial disease.

A decrease in fat amount is an important feature of CAC, where lipolysis is activated in adipocytes and the size of adipocytes is reduced. Other features, including muscle wasting, are also features of CAC; however, the crosstalk between adipocytes and other organs suggests an important role of adipocytes in systemic metabolic complications.

Tumor-derived signaling factors, systemic inflammation, and metabolic dysfunction may be responsible for CAC; however, the extracellular vesicle (EV) may also be involved. The EV, which is a nanoparticle released into the bloodstream from all cells, contains bioactive factors that act as mediators of cell-cell communication.

Since cancer cells actively release a large number of EVs throughout the body, EVs derived from cancer cells are involved in the underlying mechanism of CAC.

[Study of Induction of Lipolysis after Endocytosis in EV Derived from Pancreatic Cancer Cell]

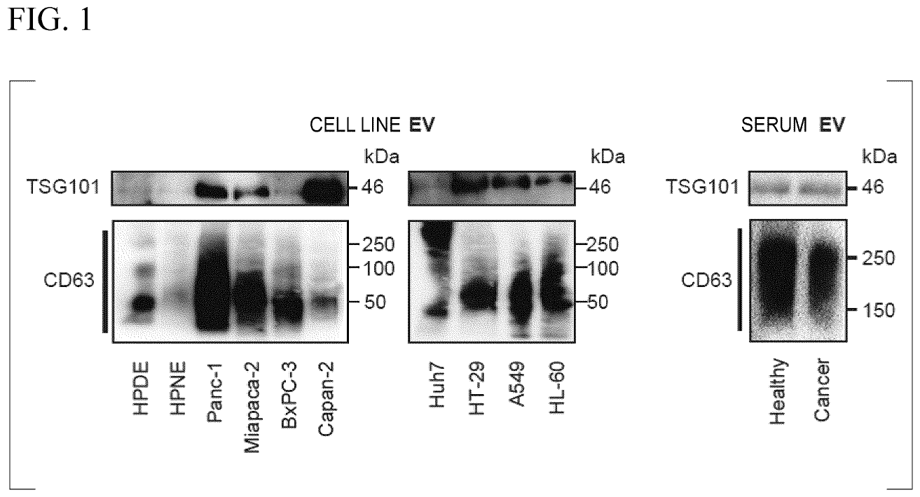

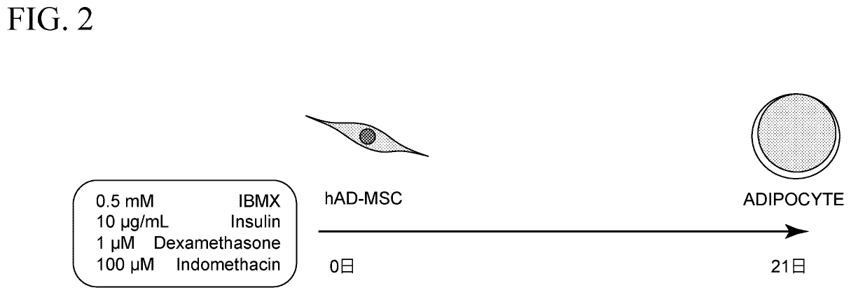

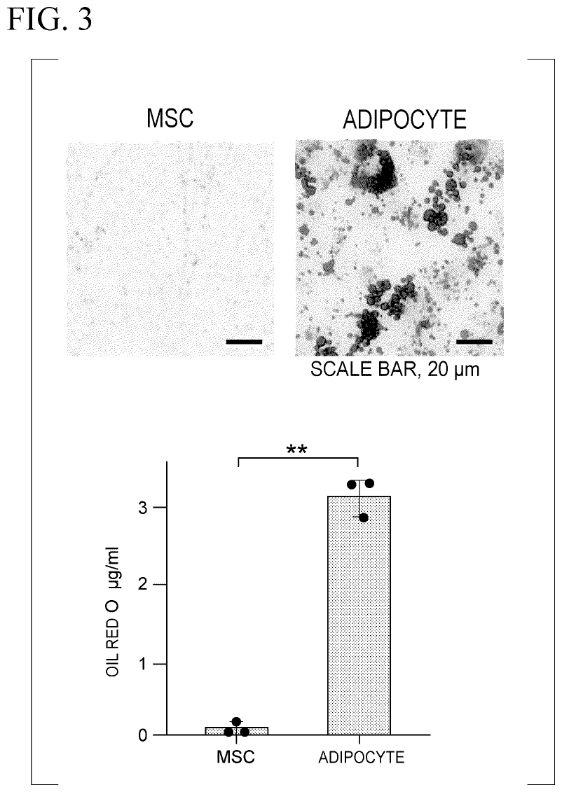

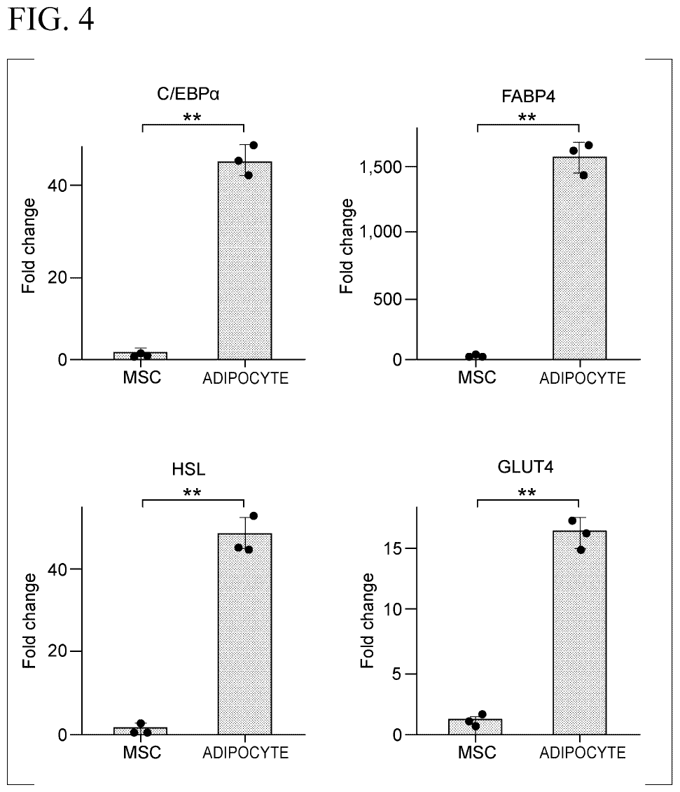

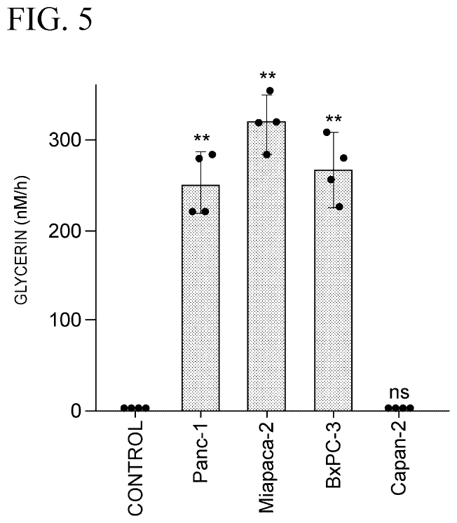

EVs having a mode diameter of 100 to 150 nm were isolated from various sources, except for EVs of about 240 nm derived from Capan-2 cells. Cancer patients had a larger number of EVs in the serum than the control. The expression of exosome markers such as TSG101 and CD63 in EVs was confirmed, whereby it was confirmed that EV isolation was appropriate (see FIG. 1). To investigate the effect of EV, mature human adipocytes were induced from human adipose-derived mesenchymal stem cells (hAD-MSCs) and subjected to an EV treatment (see FIGS. 2 to 4). Glycerin, which is an alternative marker of the lipolysis level, was released at a significantly higher level from adipocytes treated with EVs derived from Pane-1 cells. Miapapaca-2 cells, and BxPC-3 cells, but it was not observed in adipocytes treated with EVs derived from Capan-2 cells (see FIG. 5).

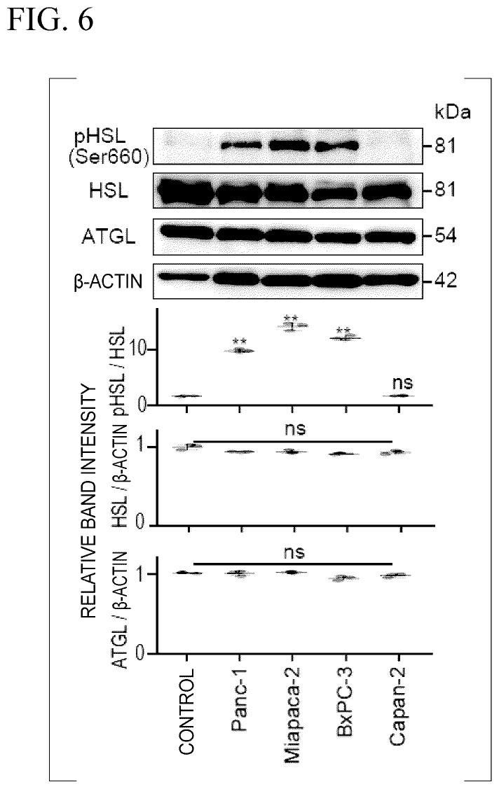

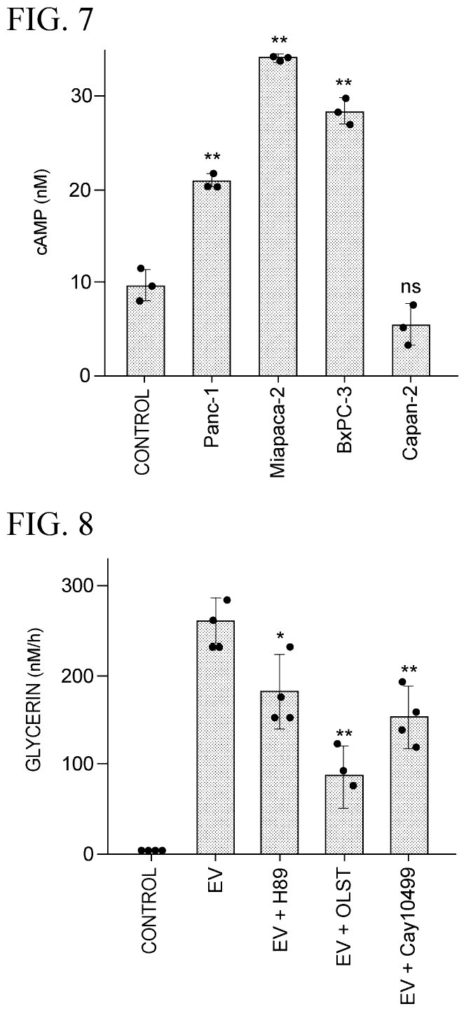

In general, the intracellular cyclic AMP (cAMP) level, the activity of protein kinase A (PKA), and the phosphorylation of Ser 660 of hormone-sensitive lipase (HSL) in adipocytes play important roles in lipolysis. Consistently, the HSL phosphorylation level and the intracellular cAMP level were upregulated significantly in adipocytes treated with EVs derived from Panc-1. Miapapaca-2, and BxPC-3 cells, which were not observed in adipocytes treated with EVs derived from Capan-2 cells (see FIGS. 6 and 7).

On the other hand, the total HSL level and the fat triglyceride lipase (ATGL) level were similar in all samples (see FIG. 6). H89 (a PKA inhibitor), Orlistat (an inhibitor for HSL and ATGL), and Cay10499 (an HSL inhibitor) significantly reduced the released glycerin level (see FIG. 8), and thus these molecules were essential for lipolysis. As a result, it was confirmed that the lipolysis of human adipocytes is induced by specific EVs through the up-regulation of the intracellular cAMP level.

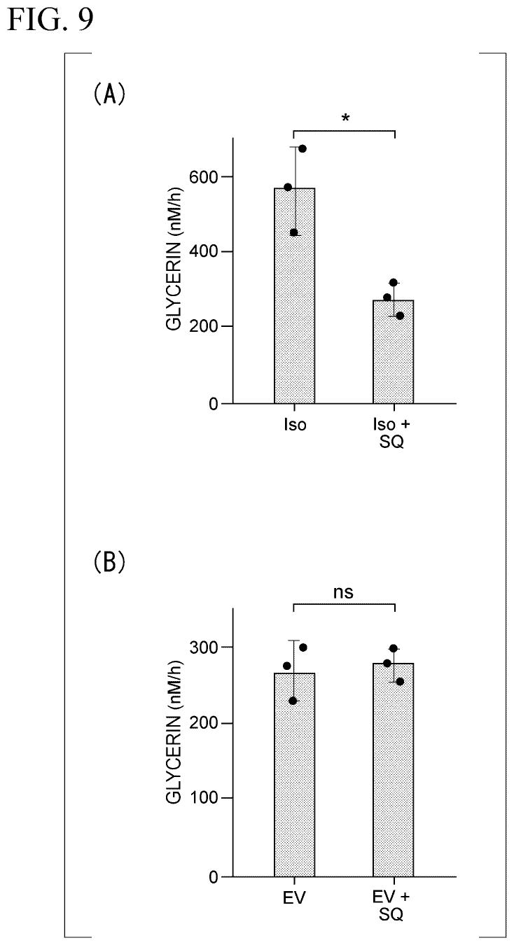

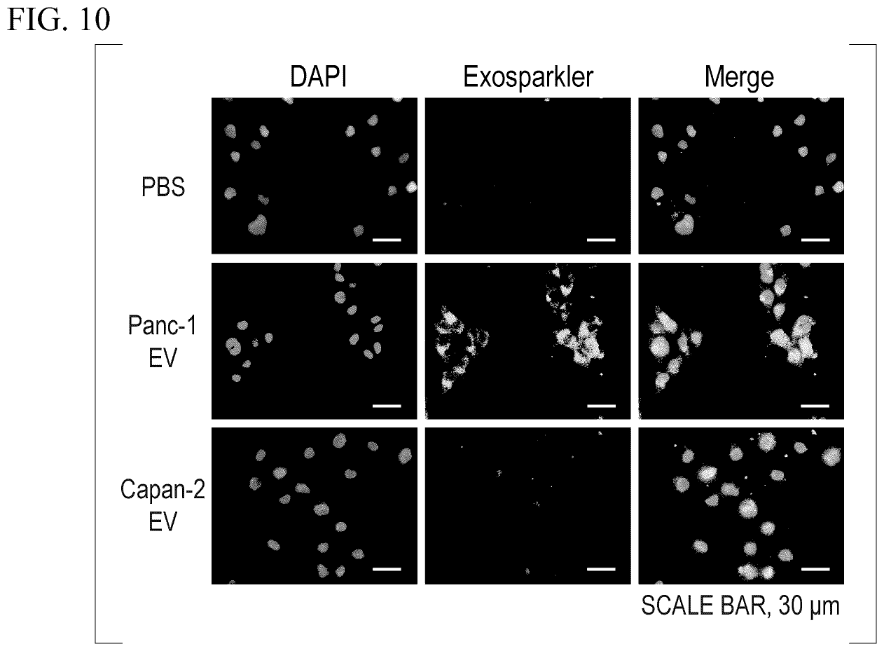

Further, the dependence of the lipolysis induced by EV, under newly synthesized cAMP, was investigated. In general, a β-stimulant such as isoproterenol activates G proteins, and it subsequently activates adenylyl cyclase, which leads to the synthesis of cAMP. As a matter of fact, isoproterenol induces lipolysis in adipocytes, which is significantly inhibited by an adenylyl cyclase inhibitor SQ22536. However, SQ22536 did not inhibit the lipolysis induced by the EVs derived from Panc-1 (see FIG. 9(A) and FIG. 9(B)). This suggests that the lipolysis induced by EV is not dependent on the newly synthesized cAMP but is dependent on cAMP in the incorporated EV. Consistently, a large number of EVs derived from Panc-1 cells were incorporated into adipocytes 5 hours after the EV treatment as compared with the number of EVs derived from Capan-2 cells (see FIG. 10).

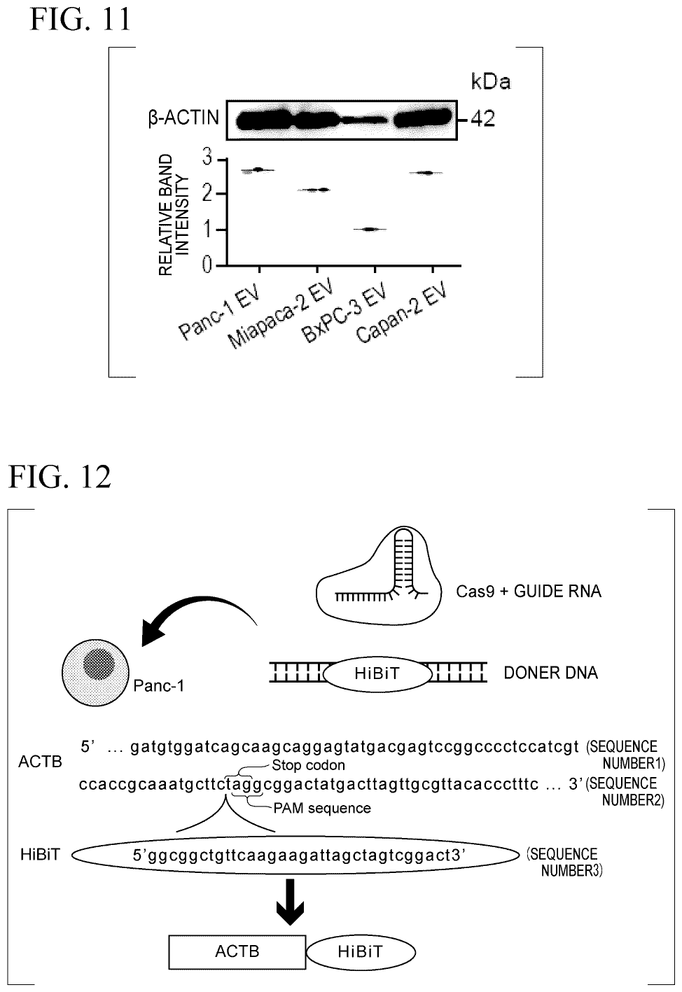

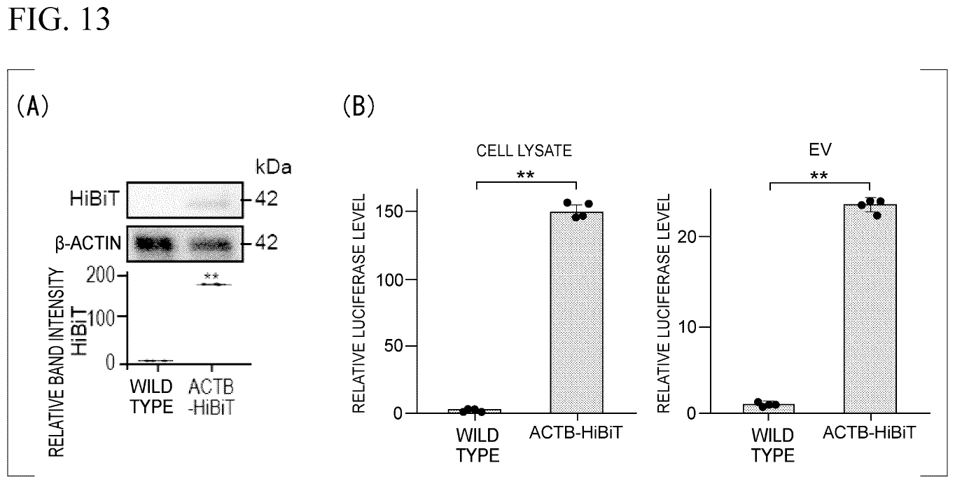

β-actin is contained as a pan-EV marker in most EVs derived from human serum and human cell lines. To monitor the EV endocytosis level, a reporter cell (Panc-1-ACTB-HiBiT cell) was established by knocking in the HiBiT peptide sequence at the 3′ terminal of the ACTB locus of Panc-1 cell (see FIGS. 11 and 12). A high luciferase activity was detected in a lysate of cells and in EVs derived from these cells, and thus it was confirmed that the β-actin-HiBiT protein is present in the EVs (see FIGS. 13(A) and 13(B).

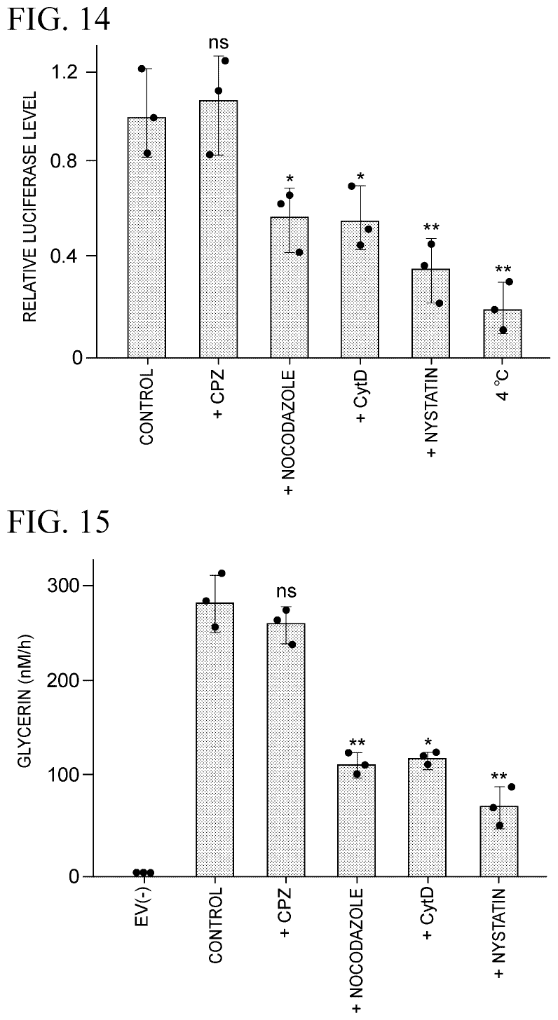

The luciferase level of the adipocyte lysate was upregualted 1 hour after the addition of EV and was similar to a level in a case where chlorpromazine, a clathrin-mediated endocytosis inhibitor, was used. However, it was significantly lower in a case where nocodazole which is a microtubule destabilizer, cytochalasin D which is an inhibitor of actin F associated with macropinocytosis, and nystatin which is an inhibitor of caveola-mediated transport were used, and in a case where all the endocytosis pathways were inhibited at 4° C. (see FIG. 14).

Therefore, the endocytosis pathway was involved in the incorporation of EV by adipocytes, except for the clathrin-mediated endocytosis. Proportionally, the level of the released glycerin decreased significantly in a case where endocytosis was inhibited (see FIG. 15).

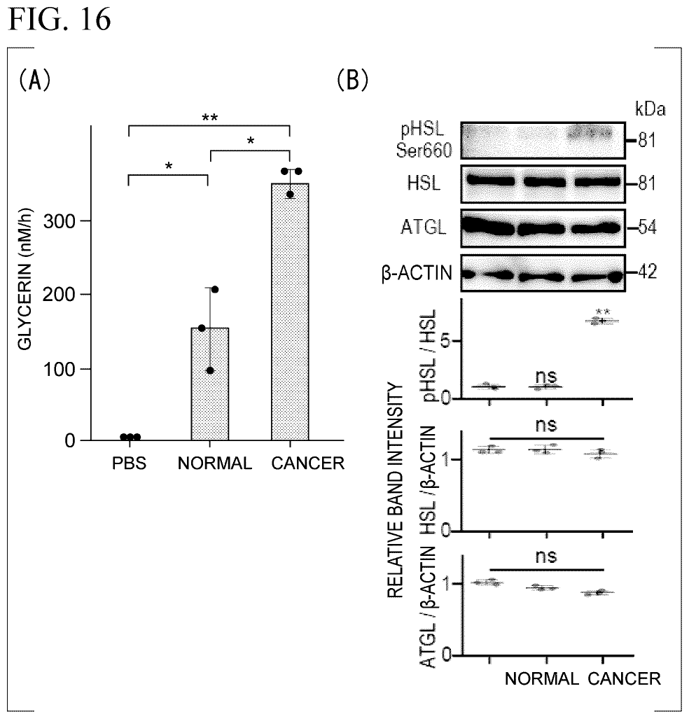

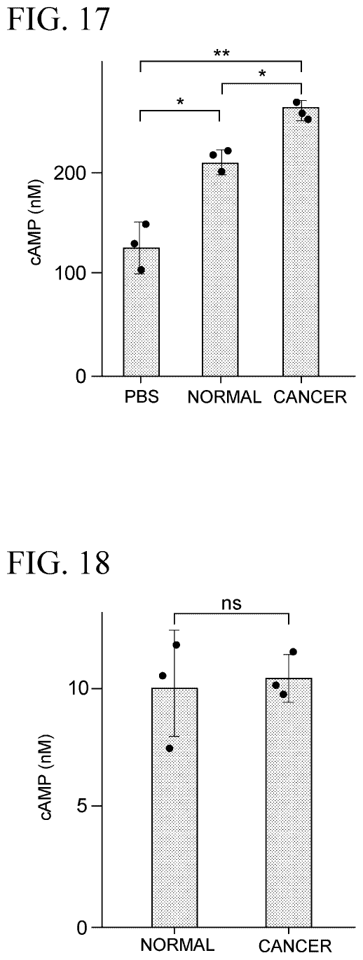

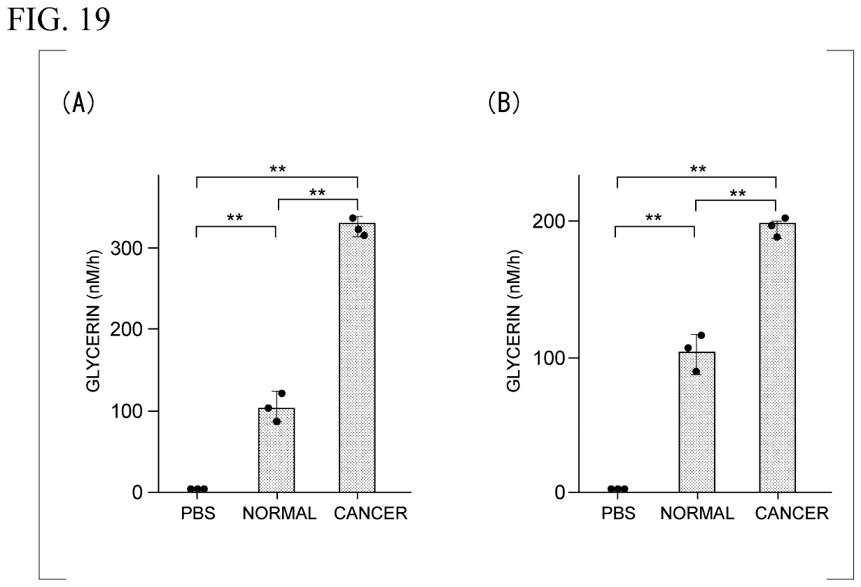

Next, it was investigated whether the EV isolated from the serum of pancreatic cancer patients would induce lipolysis in vitro. Although EVs from the healthy subjects as a control also induced a slight release of glycerin from adipocytes, EVs from pancreatic cancer patients induced lipolysis significantly (see FIG. 16(A)). Adipocytes treated with the EV of pancreatic cancer patients consistently showed a significantly higher level of HSL phosphorylation and a significantly higher level of intracellular cAMP (see FIG. 17). The observed levels were also similar in adipocytes treated with the EVs from the healthy subjects as a control; however, they were low (see FIGS. 16(A) and 16(B) and FIG. 17). In addition, no significant difference the in cAMP level was observed between the EVs themselves from the healthy subjects as control and the EVs themselves from patients (see FIG. 18). Serum from cancer patients contained more EVs than the healthy subjects as a control; however, the results were similar in a case where the number of EVs or the protein weight was adjusted (see FIG. 19(A) and 19(B)). As a result, it was confirmed that EVs in cancer patients induce lipolysis.

### [Study of Size of EV and Induction of Lipolysis in Pancreatic Cancer Patient]

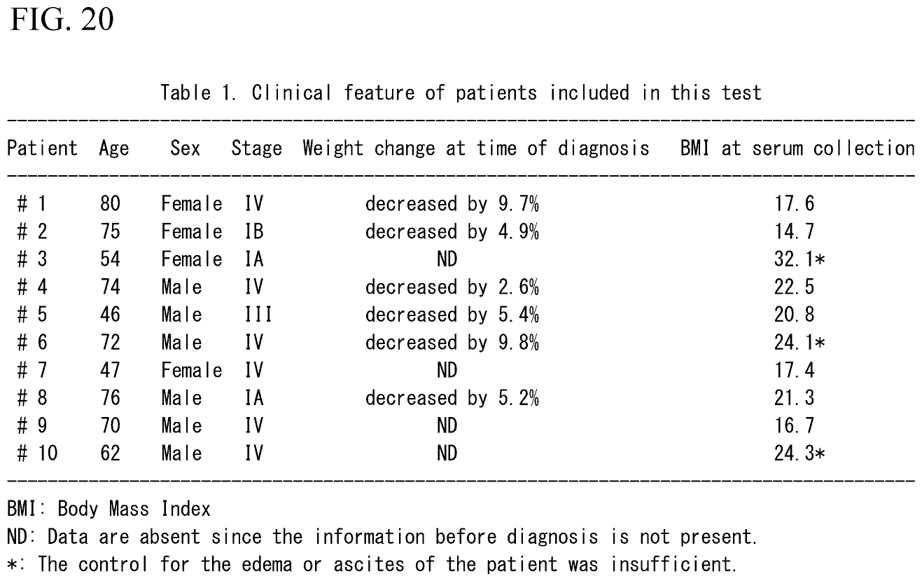

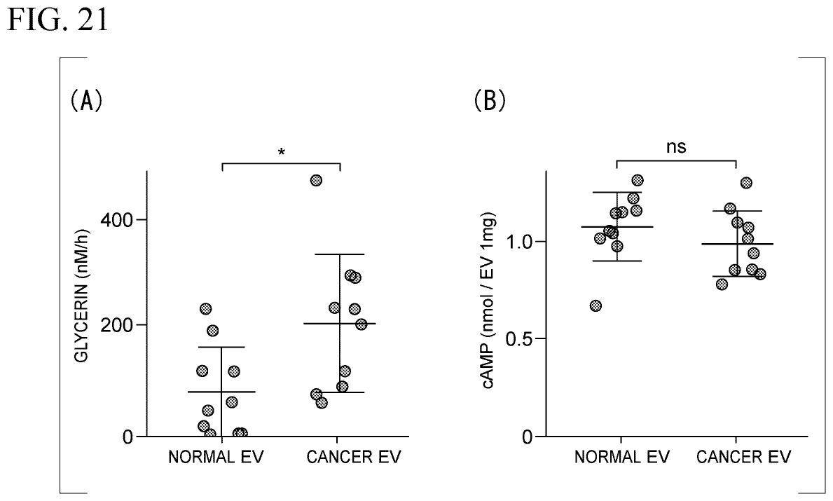

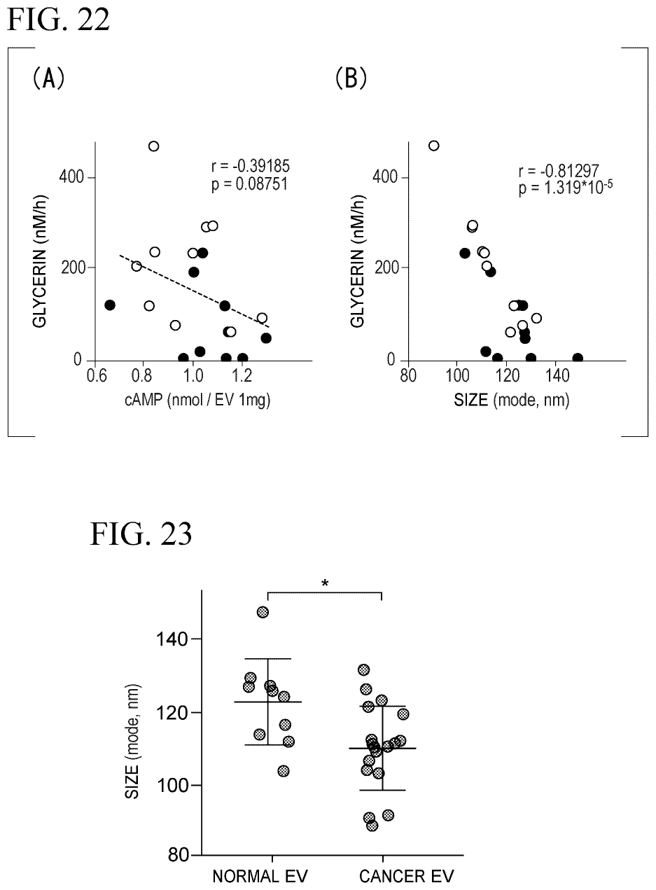

Next, as a result of investigating the EVs in the individual case, body weight was significantly reduced in almost all pancreatic cancer cases (see FIG. 20). Although the EV from the control example also induced lipolysis, the EVs from pancreatic cancer patients induced lipolysis significantly (see FIG. 21(A)). There was no significant difference in the cAMP level of EV between donors (see FIG. 21(B)). Further, the cAMP levels of the EVs from the control and the cancer cases were not correlated with the lipolysis levels (see FIG. 22(A)). Rather, the lipolysis level was significantly correlated with the differential sizes of the EVs in both the control and the pancreatic cancer cases (P=1.32×10⁻⁵, r=−0.81297) (see FIG. 22(B)). Importantly, the serum of pancreatic cancer patients was significantly small as compared with that of the healthy control (see FIG. 23). These results suggest that the degree of induction of lipolysis in human adipocytes increases as the EV size decreases.

### [Study of Incorporation of EV by Adipocytes]

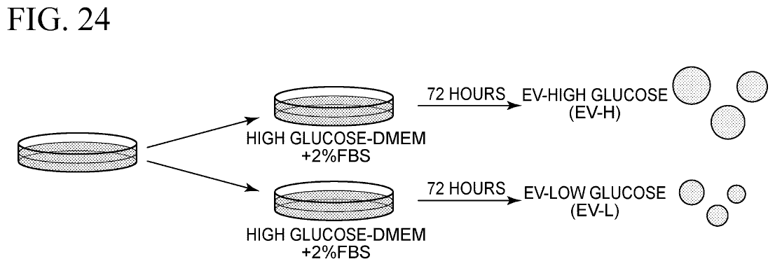

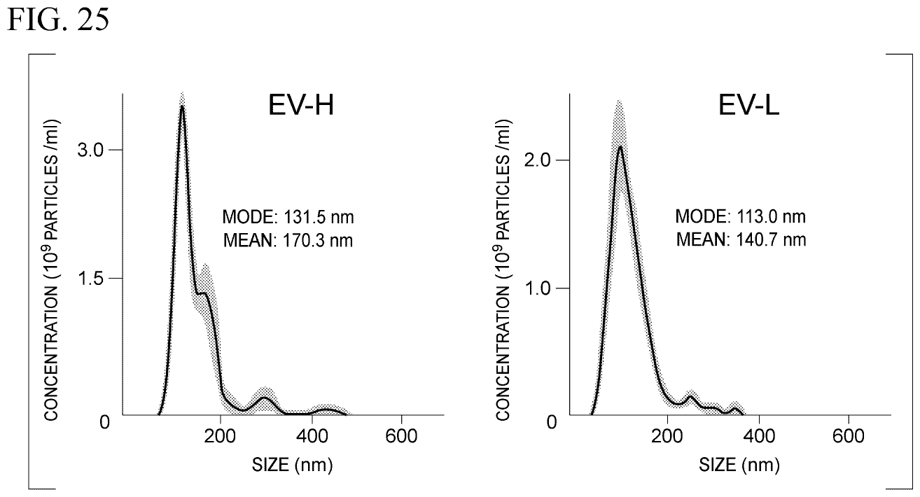

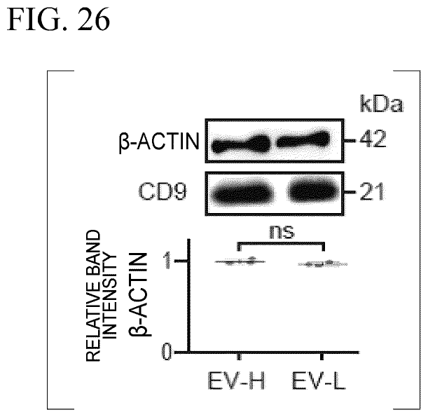

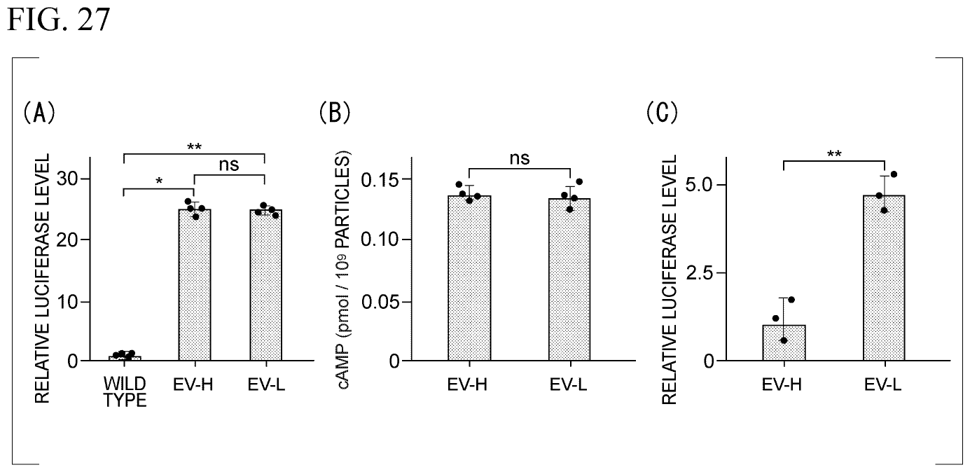

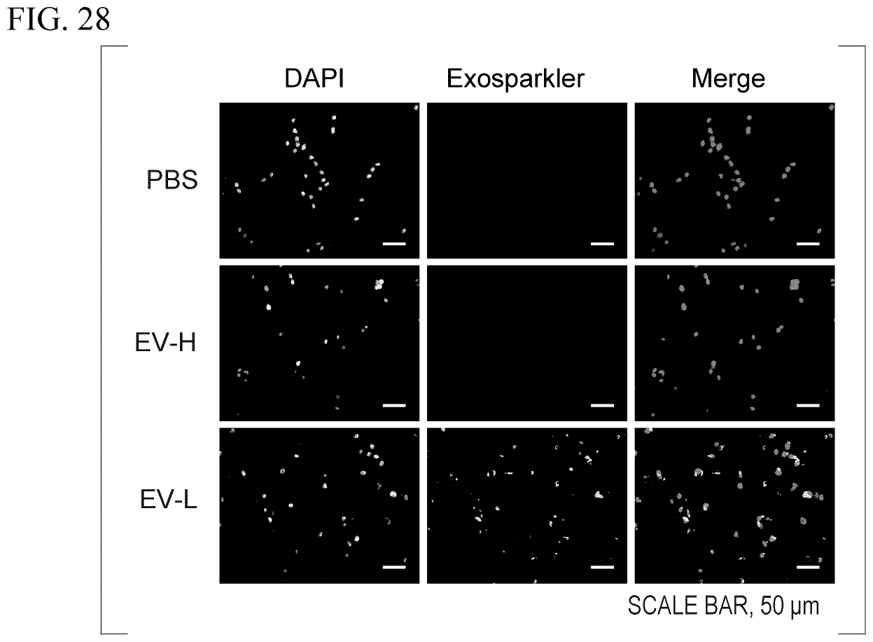

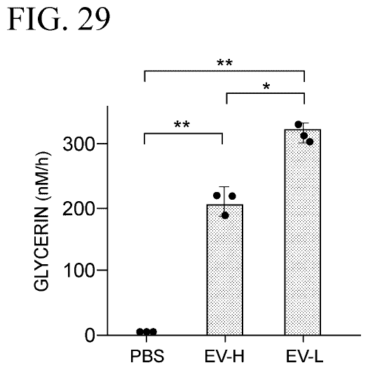

Two methods were applied to change the EV size. EVs (EV-Low: EV-L) from cells cultured in a low glucose culture medium were significantly smaller than EVs (EV-High: EV-H) from cells cultured in a high glucose culture medium (see FIGS. 24 and 25). In EVs derived from Panc-1-ACTB-HiBiT cells cultured in a low glucose or high glucose culture medium, there was no significant difference in the β-actin protein, the HiBiT peptide, or the cAMP level (see FIGS. 26, 27 (A), and 27 (B)), and EV-H and EV-L were similar except for size. However, the luciferase level from the lysate of the adipocytes treated with EV was significantly high in a case where EV-L was used (see FIG. 27(C)). This suggests that the incorporation of EV increases as the size thereof decreases. Similarly, a larger number of the fluorescently labeled EV-Ls were incorporated by adipocytes (see FIG. 28), indicating a significant increase in the induction of lipolysis (see FIG. 29).

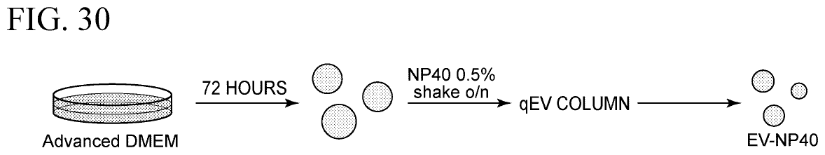

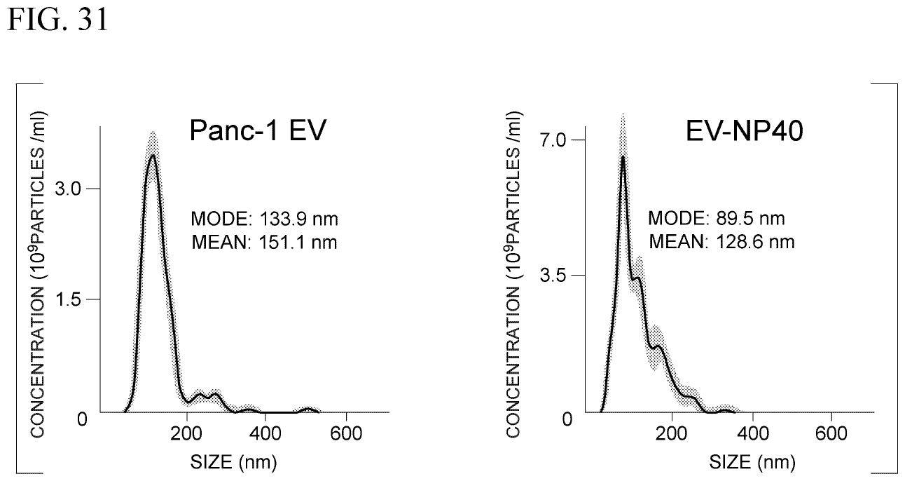

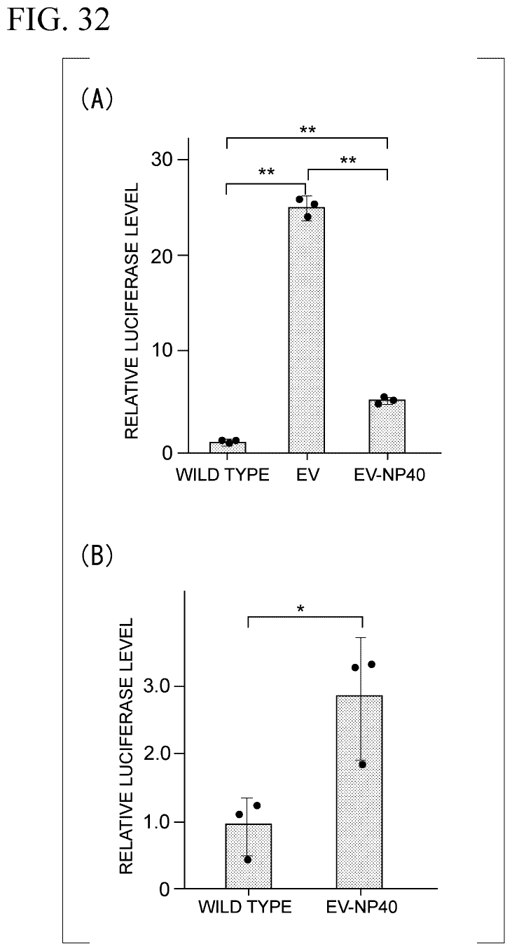

As a second method to change the EV size, NP40 was added to the isolated EV at a final concentration of 0.5% (see FIG. 30). The addition of NP40 partially damaged the lipid bilayer of EV and reduced the size of EV (EV-NP40) (see FIG. 31). In addition, EV-NP40 also had a significantly low luciferase level (see FIG. 32(A)). This is due to the loss of the β-actin-HiBiT protein due to damage to the lipid bilayer. However, the luciferase level of the lysate of the adipocytes treated with the same number of EVs was significantly high in a case where EV-NP40 was used (see FIG. 32(B)). From these results, it has been confirmed that the incorporation of EV increases as the site thereof decreases.

[Study of Size of EV Derived from Cancer Cell in Patient Serum]

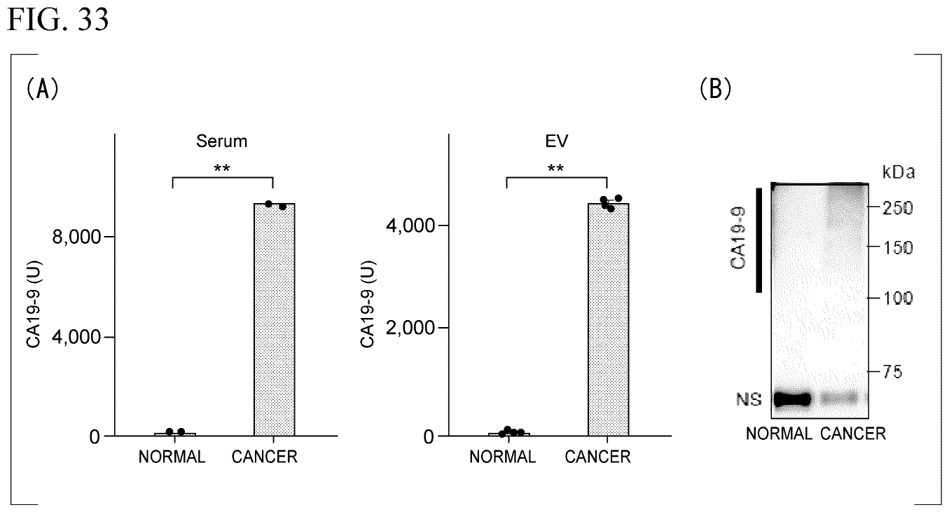

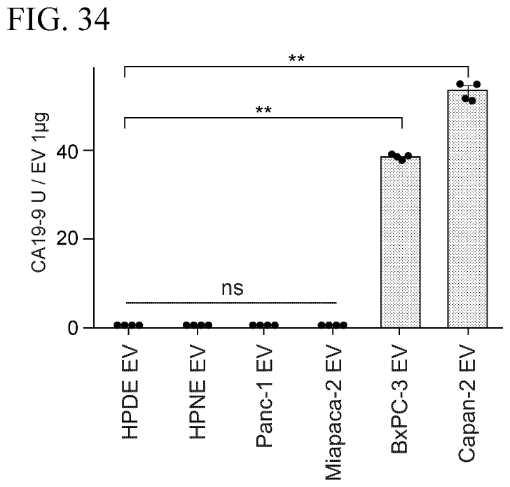

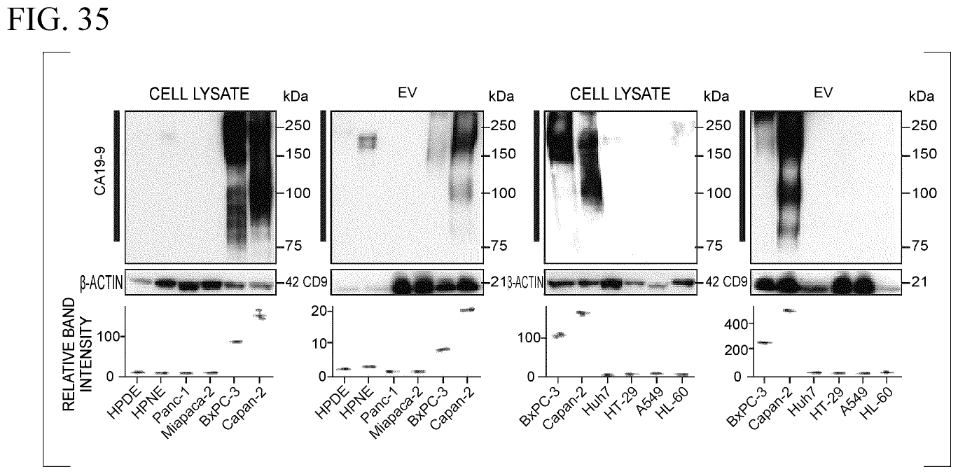

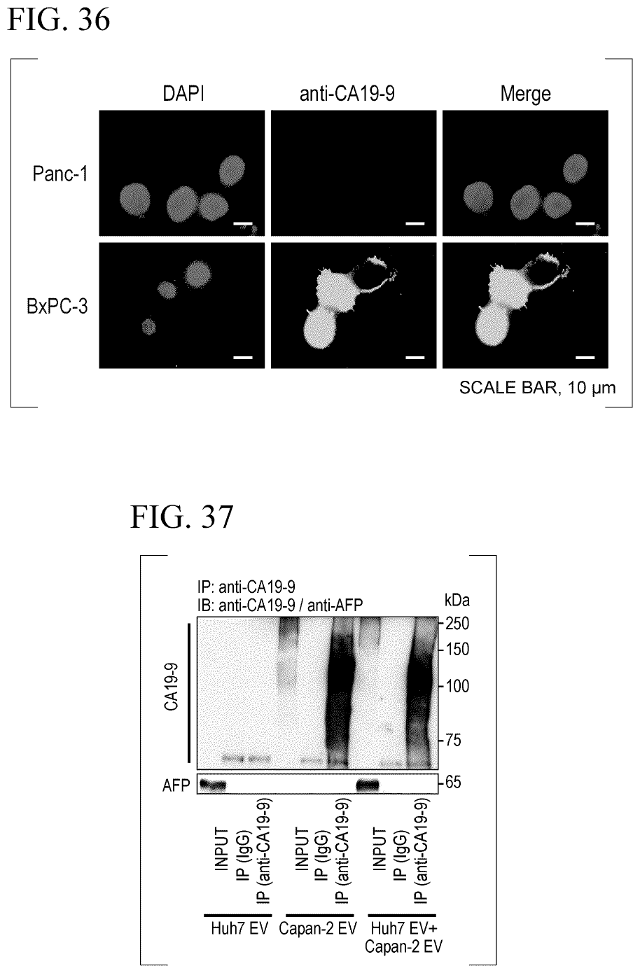

To characterize EVs derived from cancer cells, a specific population of EVs was isolated by immunoprecipitation. The carbohydrate antigen 19-9 (CA19-9) is a cell surface sugar chain. CA19-9 is also produced in the normal pancreas. However, the synthesis thereof is significantly increased by abnormal sialylation in the pancreas. Since EVs are produced by recycling cell membranes, CA19-9 should reside on the surface of EVs from the CA19-9 positive cells. As expected, CA19-9 was abundantly contained in the bulk serum and EVs of pancreatic cancer patients (see FIG. 33). To establish a method for isolating CA19-9 positive EVs, CA19-9 negative EVs derived from normal human pancreatic ductal epithelial cells (HPDE and HPNE), and CA19-9 negative pancreatic cancer cells (Panc-1 and Miapaca-2), and CA19-9 negative EVs derived from pancreatic cancer cells (BxPC- and Capan-2) (see FIGS. 34 to 36) were used. The CA19-9 positivity in EV was proportional to the expression of CA19-9 in cells including the hepatocellular cancer cell (Huh7), the colorectal carcinoma cell (HT-29), the lung cancer cell (A549), and the leukemia cell (HL-60) (see FIG. 35).

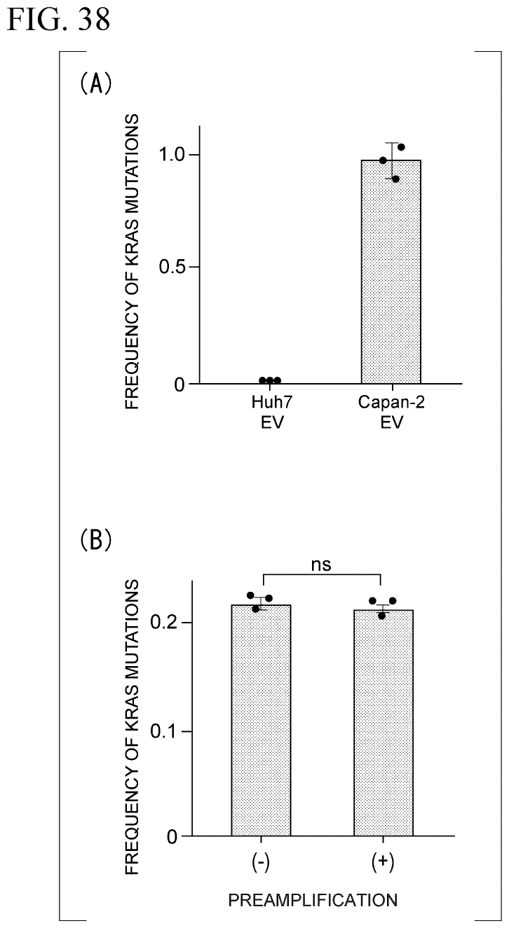

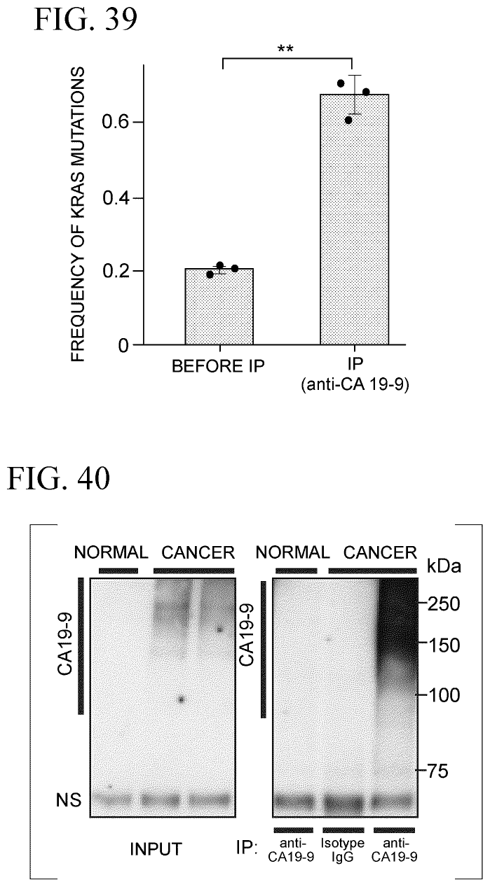

To check the rationale of the IP method targeting CA19-9, EVs derived from Huh7 cells (CA19-9 negative), EVs derived from Capan-2 cells (CA19-9 positive), and a mixture thereof were subjected to immunoprecipitation. The Western blotting of the IP sample confirmed a high concentration of the CA19-9 positive EV. The specificity was checked by using an isotype IgG for IP and assaying AFP (an abnormal protein that is used as a marker for hepatocellular cancer) detected only in the EVs derived from Huh7 cells (see FIG. 37). Since almost all pancreatic cancer patients have a KRAS mutant allele in the cancer cells thereof, the frequency of KRAS mutations in mRNA of EV was investigated in order to verify the specificity of the IP method. The frequency of KRAS mutations in the input sample (before IP) in EVs derived from Huh7 cells (carrying only wild-type KRAS) and EVs derived from Capan-2 cells (carrying a heterozygous KRAS mutation), where the number of Huh7 cells and the number of Capan-2 cells were the same, and the IP sample was investigated (see FIG. 38(A)). After confirming that preamplification did not affect the results (see FIG. 38(B), the frequency of KRAS mutations was confirmed to be higher in the IP sample than in the input sample (see FIG. 39). This indicates that EVs derived from the CA19-9 positive Capan-2 cells are concentrated.

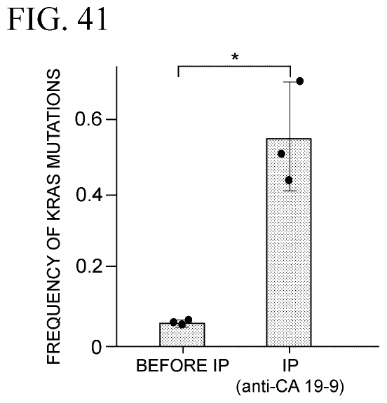

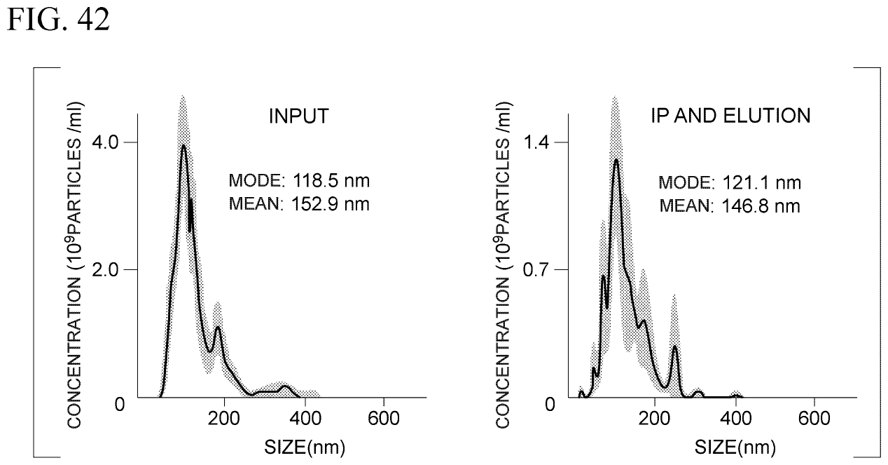

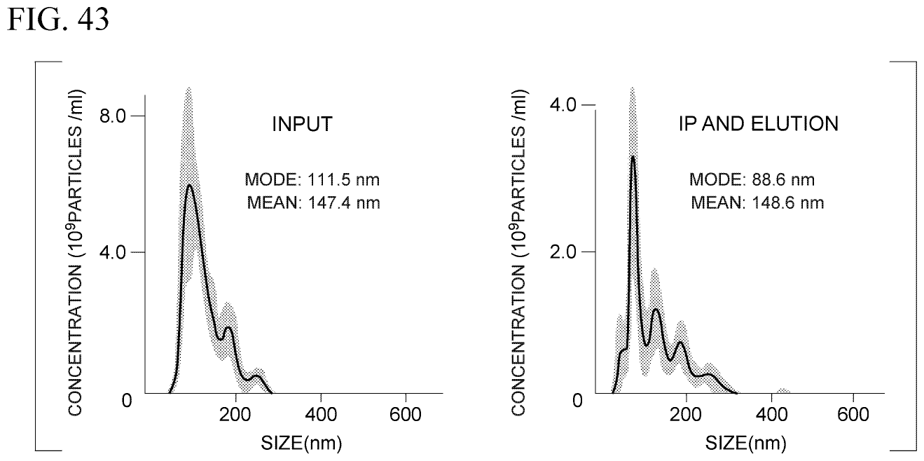

Similar to the EVs derived from cells, the CA19-9 positive EVs were concentrated by using EVs derived from the serum of pancreatic cancer patients (see FIG. 40). The frequency of KRAS mutations was significantly higher in EVs concentrated by IP (see FIG. 41). After confirming that IP and elution did not affect the EV size distribution (see FIG. 42), it was confirmed that the EVs derived from cancer cells were significantly smaller than the total EVs from the serum of pancreatic cancer patients (see FIG. 43).

[Study of Induction of Lipolysis in EV Derived from Pancreatic Cancer Cell]

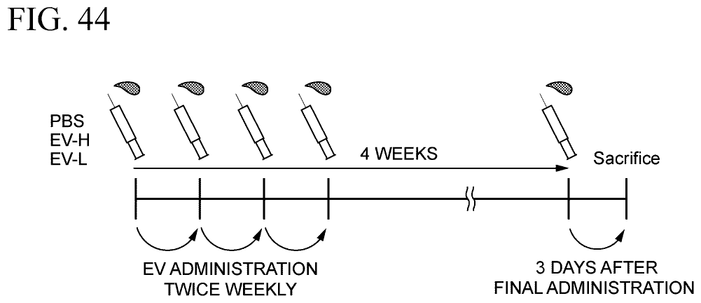

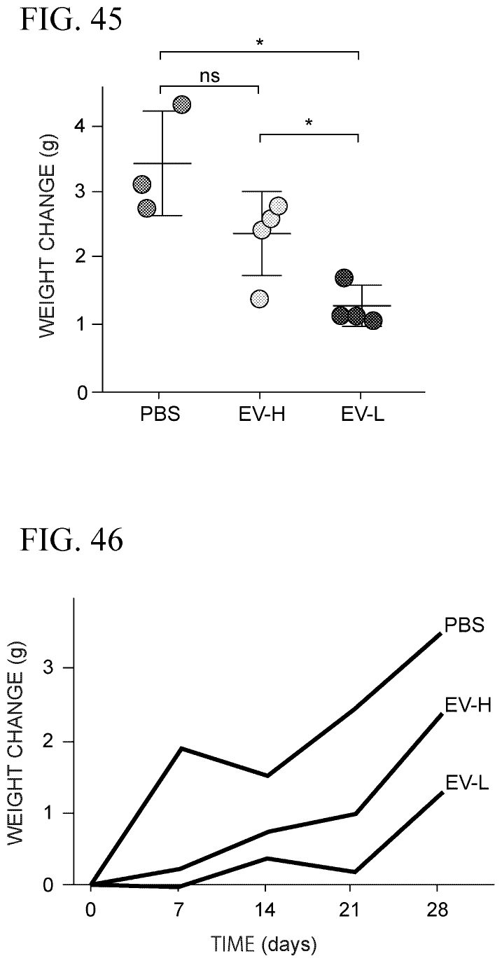

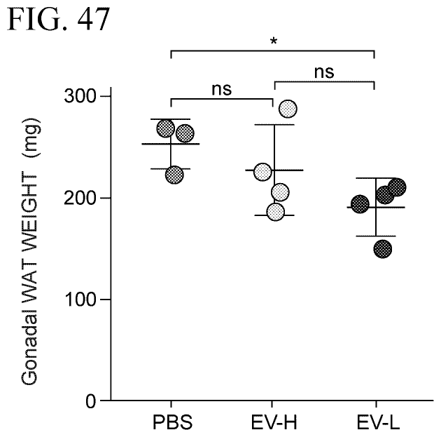

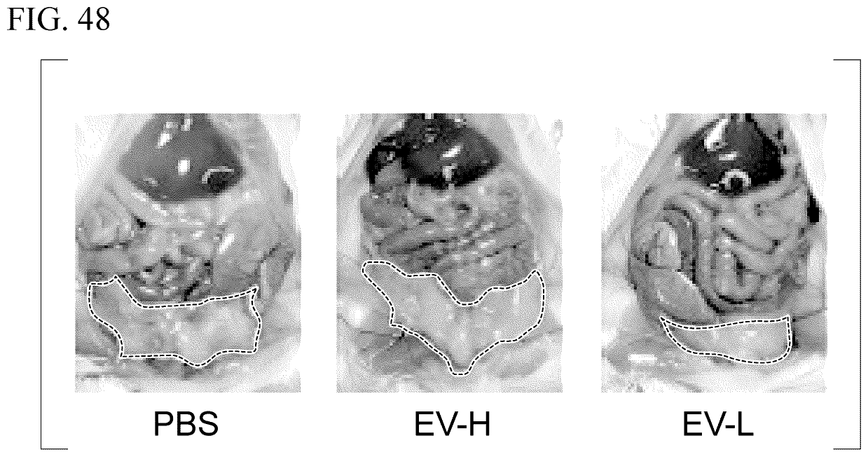

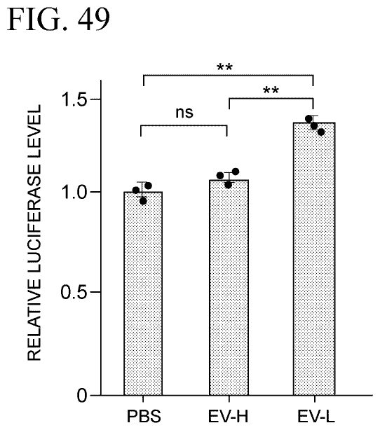

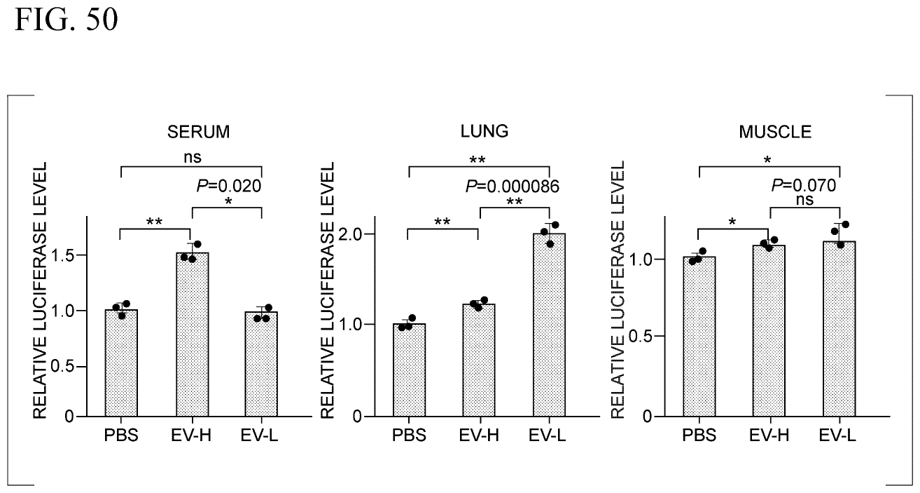

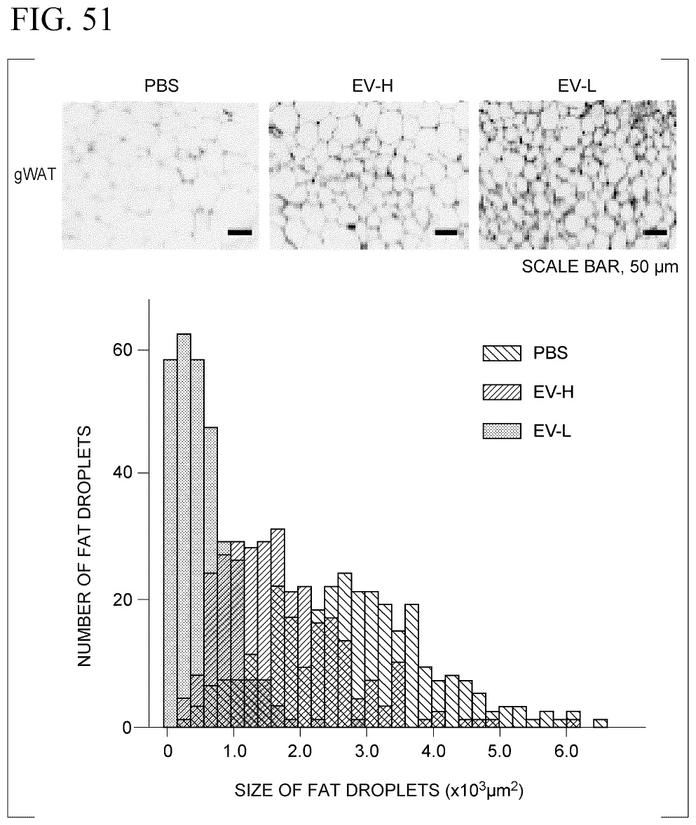

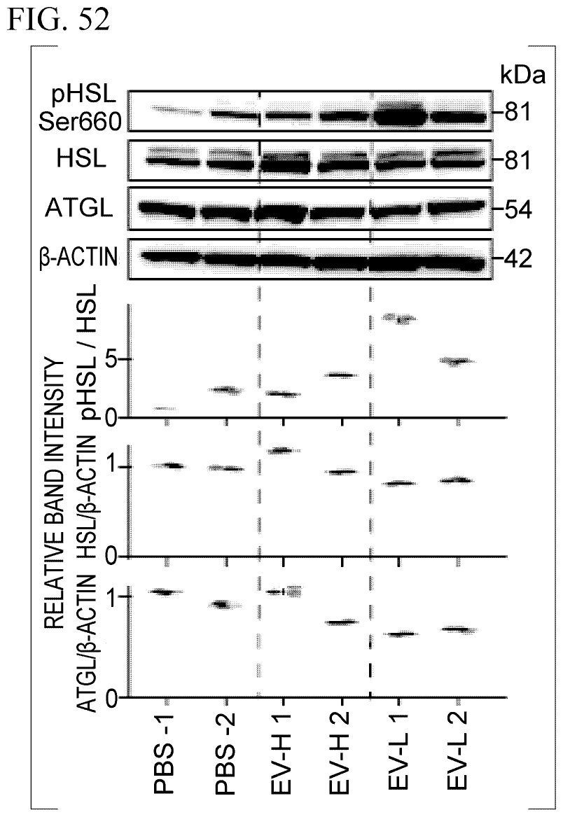

To confirm the above results in vivo, mice were intravenously administrated with the EVs derived from Panc-1-ACTB-HiBiT cells (EV-H and EV-L), which had different sizes (see FIG. 44). Mice treated with larger EVs (EV-H) showed a slight reduction in the body weight increase at 4 weeks after administration as compared with the control, whereas mice treated with smaller EVs (EV-L) showed a 63.6% reduction in the body weight increase as compared with the control (see FIG. 45). Mice treated with EV-L showed a small body weight increase (see FIG. 46). The gonadal white adipose tissue (gWAT) of the mice treated with EV-L had a significantly small weight and a significantly small size (see FIGS. 47 and 48). Consistent with the in vitro results, more EV-L was incorporated into the gWAT, as estimated by the HiBiT luciferase activity (see FIG. 49). EV-L was taken up more by gWAT. The smaller EVs (EV-L) were incorporated more into the lungs than the EV-H, but not into the muscles, and the EV-H level in the serum was high after 3 days (see FIG. 50). Fat droplets having a significantly small size were observed in the gWAT of mice treated with EV-L (see FIG. 51). Consistently, the phosphorylation at Ser660 of HSL in the gWAT was higher in mice treated with EV-L (see FIG. 52). As a result, the smaller the EVs, the more EVs were incorporated into adipocytes, leading to greater induction of lipolysis. Cancer cells rely on glycolysis for energy production, and thus the tumor microenvironment of solid cancers generally has a low glucose level. The fewer EVs derived from pancreatic cancer cells may be due to a low glucose level in the tumor microenvironment of the pancreatic cancer tissue. This indicates that, together with the fact that more EVs are released by cancer cells, EVs derived from tumors in pancreatic cancer mediate systemic induction of lipolysis as a feature of the cancer cachexia.

### [Study of Detection of Surface Proteins of EV Using Oligo DNA]

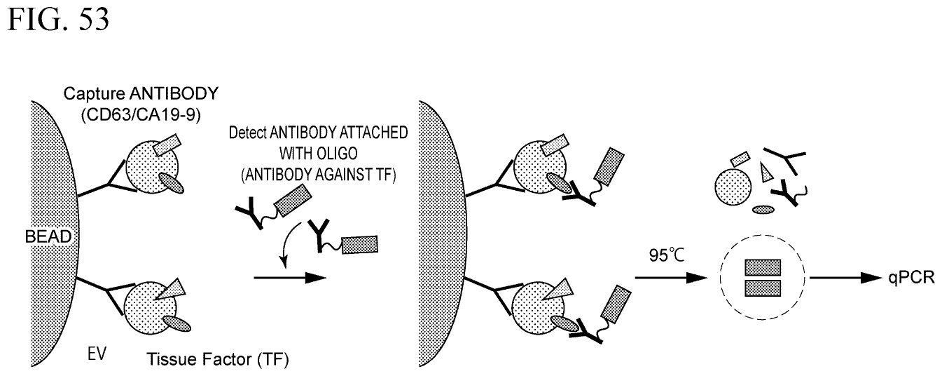

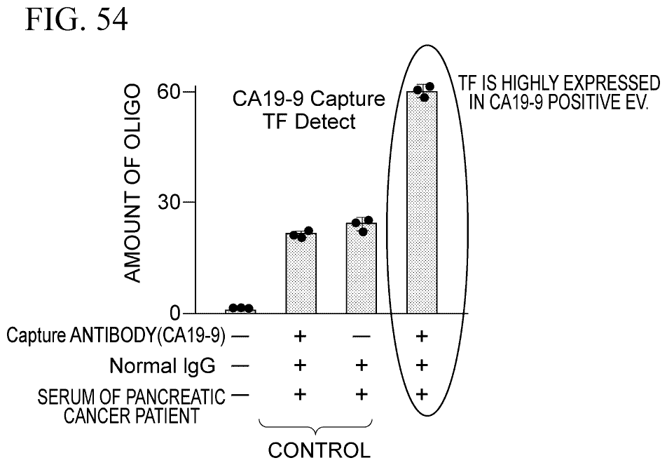

Beads, to which an anti-CA19-9 antibody was bound, and EV were incubated to form a first complex between the beads, to which an anti-CA19-9 antibody was bound, and the EV. Next, an oligo to which an anti-Tissue Factor (TF) antibody was bound was added to the first complex, and the EV bound to the beads was bound to the anti-TF antibody to form a second complex. This second complex was subjected to quantitative PCR (see FIG. 53). The amount of the amplified product in this quantitative PCR is proportional to the amount of the Detect antibody bound to the original EV, that is, the amount of the surface antigen (TF in this case) expressed on the EV. The results are shown in FIG. 54. It was confirmed that TF is highly expressed in the CA19-9 positive EV.

### INDUSTRIAL APPLICABILITY

According to the present embodiment, it is possible to diagnose cancer in a highly accurate and noninvasive manner.

1. A method for isolating and concentrating an extracellular vesicle group derived from a cancer cell, the method comprising:
bringing a substance having an affinity for a carbohydrate antigen CA19-9 into contact with an extracellular vesicle group which has been isolated from blood derived from a patient, and isolating and concentrating an extracellular vesicle group derived from a cancer cell, from the extracellular vesicle group.

2. The method for isolating and concentrating an extracellular vesicle group derived from a cancer cell according to claim 1, wherein the substance having an affinity for a carbohydrate antigen CA19-9 is an anti-CA19-9 antibody.

3. The method for isolating and concentrating an extracellular vesicle group derived from a cancer cell according to claim 1, wherein the substance having an affinity for a carbohydrate antigen CA19-9 further includes a carrier.

4. The method for isolating and concentrating an extracellular vesicle group derived from a cancer cell according to claim 3, wherein the carrier is a bead.

5. An analysis method comprising:
analyzing cancer-associated genes in an extracellular vesicle group derived from a cancer cell by the method according to claim 1, for a genotype of the patient.

6. A drug selection method comprising:
selecting a drug to be administered, based on the genotype of the patient, which is analyzed using the analysis method according to claim 5.

7. A kit for isolating and concentrating an extracellular vesicle group derived from a cancer cell, from an extracellular vesicle group isolated from blood derived from a patient, the kit comprising:
a substance having an affinity for a carbohydrate antigen CA19-9.

8. The kit according to claim 7, wherein the substance having an affinity for a carbohydrate antigen CA19-9 is an anti-CA19-9 antibody.

9. The kit according to claim 7, wherein the substance having an affinity for a carbohydrate antigen CA19-9 includes a carrier.

10. The kit according to claim 9, wherein the carrier is a bead.

11. An anti-cancer agent comprising:
as an active ingredient, an anti-CA19-9 antibody that neutralizes a function of a carbohydrate antigen CA19-9.
